(19) United States
(12) Patent Application Publication
(10) Pub. No.: US 2024/0140974 A1
(43) Pub. Date: May 2, 2024

(54) PLATINUM/PEPTIDE/CUCURBITURIL COMPLESES: WELL-DEFINED ARCHITECTURES BUILT BY SUPRAMOLECULAR SELF-SORTING FOR THE TARGETING OF CYSTEINE PROTEASES

(71) Applicant: Ohio University, Athens, OH (US)

(72) Inventor: Eric Masson, Athens, OH (US)

(21) Appl. No.: 18/554,869

(22) PCT Filed: May 10, 2022
(86) PCT No.: PCT/US2022/028534

§ 371 Date: Oct. 11, 2023

Related U.S. Application Data
(60) Provisional application No. 63/186,493, filed on May 10, 2021.

(51) Int. Cl. C07F 15/00; A61K 47/69; C07K 7/06; C12N 9/50

(52) U.S. Cl. CPC C07F 15/0093; A61K 47/6949; C07K 7/06; C12N 9/506

(57) ABSTRACT

The present disclosure concerns cucurbit[n]uril (CB) complexes of cucurbit[8]uril (CB[8]) and/or cucurbit[7]uril (CB[7]) that secure or host platinum conjugated terpyridines. The platinum center can further secure a protein through the thiol of a cysteine or an imidazole of a histidine therein. Additional CBs can secure the peptide through phenyl side chains. CB[8] will secure a dimer of platinum-terpyridines in a head-to-head alignment or a peptide dimer through a head-to-tail alignment. The application of multiple CBs allows for varying CB complex geometries. Also contemplated are methods of using the platinum-terpyridines to selectively bind cysteine rich proteins, such as cysteine proteases.

### RELATED APPLICATION

This invention claims priority to U.S. Provisional Application Ser. No. 63/186,493, filed May 10, 2021, which is hereby incorporated by reference herein in its entirety.

### FEDERAL SUPPORT

This invention was made with government support National Science Foundation (grant CHE-1507321), the American Chemical Society Petroleum Research Fund (grant 56375-ND4). The Government has certain rights in the invention.

### TECHNICAL FIELD

The invention relates to the targeted binding and complexing between Curcurbit[8]uril (CB[8]) supramolecular complexes and Curcurbit[7]uril (CB[7]) supramolecular complexes with cysteine containing peptides. The complexes are assembled with secured Pt centers to couple to available thiols in cysteine residues. The complex's binding of such offers novel approaches to neutralizing active thiol groups in peptides and proteins.

### SUMMARY

The present disclosure concerns cucurbit[n]uril hosted complexes with platinum-terpyridine hosts conjugated to peptides or proteins. In some aspects, the present disclosure concerns a cucurbituril (CB) peptide complex that includes a first CB of cucurbit[8]uril (CB[8]) or cucurbit[7]uril (CB[7]), a first platinum terpyridine (Pt-tpy) and a first peptide. In some aspects, the first peptide is bound to the first Pt-tpy. In further aspects, the first peptide is bound to the first Pt-tpy through a cysteine or histidine residue. In certain aspects, the cysteine or histidine residue is at a terminus of the first peptide. In some aspects, the CB is CB[8].

In some aspects, the Pt-tpy-peptide of the CB complex is the following:

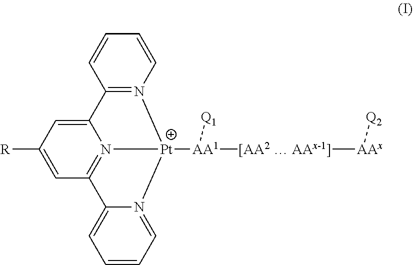

where: R is phenyl or substituted phenyl; each AA is an amino acid of a peptide; AA¹ is an amino acid bound to a platinum metal center; AA^{X} is a terminal or distal amino acid of the peptide; x is from 2 to 5000; and Q₁ and Q₂ are independently either non-existent or at least one additional amino acid of the peptide. In some aspects, the complex includes at least one curcurbituril CB[n], where n is from 5 to 10. In some aspects, each curcurbituril CB[n] is circumposed about a head portion or a tail portion of the at least one platinum-terpyridine-peptide complex. In some aspects, the head portion of the at least one platinum-terpyridine-peptide complex comprises group R of the at least one platinum-terpyridine-peptide complex; and the tail portion of the at least one platinum-terpyridine-peptide complex comprises the terminal or distal amino acid AA^{X}. In some aspects, each AA¹ is a cysteine or histidine residue. In some aspects, x is from 2 to 5. In some aspects, amino acid AA^{X} is phenylalanine or tyrosine In certain aspects, AA^{X} is a terminal amino acid. In some aspects, R is an o-, m-, or p-tolyl, a phenol, or a halogenated phenyl. In some aspects, R is a chlorinated phenyl, a dichlorinated phenyl, a fluorinated, or a difluorinated phenyl.

In some aspects, the Pt-tpy of the complex is 4′-(3,5-difluorophenyl)-2,2′:6′,2″-terpyridine platinum(II) or 4′-(p-tolyl)-2,2′:6′,2″-terpyridine platinum (II).

In some aspects, the CB peptide includes two platinum-terpyridine-peptide complexes and one curcurbituril CB[n], in which the curcurbituril CB[n] is circumposed about both head portions of the two platinum-terpyridine-peptide complexes. In some aspects, the CB is CB[8]. In further aspects, the CB[8] secures a head-to-head (HH) dimer of the first and second platinum-terpyridine-peptide complexes. In some aspects, the second peptide of the second Pt-tpy-peptide complex is identical in amino acid sequence to the first peptide of the first Pt-tpy-peptide complex. In some aspects, the first Pt-tpy and the second Pt-tpy are identical.

In some aspects, the CB peptide complex may further include a second CB of CB[8] or CB[7]. In some aspects, the second CB is secured to the first peptide through a phenylalanine or tyrosine residue. In some aspects, the first CB is CB[8] and the first CB secures both a first platinum-terpyridine-peptide complexes and a second platinum-terpyridine-peptide complexes. In some aspects, the second CB is CB[8]. In some aspects, the second CB secures at least the second peptide. In some aspects, the first and second CBs secure two different amino acids of the same peptide.

In some aspects, the CB complex may further include a third CB. In some aspects, the complex includes two platinum-terpyridine-peptide complexes and three CB[n], in which a first CB[n] is circumposed about both head portions of the two platinum-terpyridine-peptide complexes, a second CB[n] is circumposed about the tail portion of one of a first of the two platinum-terpyridine-peptide complexes, and a third CB[n] is circumposed about the tail portion of a second of the two platinum-terpyridine-peptide complexes. In some aspects, the second CB is CB[7] and the third CB is CB[7]. In some aspects, the second CB secures the first peptide and the third CB secures the second peptide.

In some aspects, the present disclosure concerns methods for preparing the CB peptide complexes herein through conjugating the first peptide to the first Pt-tpy and subsequently adding an equivalent of the first CB thereto. In some aspects, the methods may include adding a further equivalent of CB[8] or CB[7]. In certain aspects, the further equivalent is of about 0.5 to the concentration of the first Pt-tpy. In certain aspects, the further equivalent is of about 2 to the concentration of the first Pt-tpy. In some aspects, the first peptide conjugates to the first Pt-tpy by displacing a halogen ligand.

In some aspects, the present disclosure concerns a method of binding a peptide or a protein by incubation of a CB Pt-tpy complex of with a peptide in a solution. In some aspects, the peptide includes at least one cysteine and/or histidine residue. In some aspects, the CB Pt-tpy includes a Pt-tpy secured or hosted in a CB. In some aspects, the CB complex includes CB[8]. In some aspects, the CB Pt-tpy include a secured Pt-tpy dimer. In some aspects, the CB complex includes CB[7].

In some aspects, the peptide includes Mpro of SARS-CoV-2. In some aspects, the solution is in vitro. In some aspects, the solution is in vivo.

In some aspects, the present disclosure concerns a cucurbituril (CB) peptide complex that includes a first CB of cucurbit[8]uril (CB[8]) or cucurbit[7]uril (CB[7]), a first platinum terpyridine (Pt-tpy) and a cysteine protease. In some aspects, the cysteine protease is conjugated to the Pt-tpy. In some aspects, the cysteine protease is conjugated through a cysteine and/or a histidine residue. In some aspects, the cysteine protease is MPro of severe acute respiratory syndrome coronavirus 2 (SARS-CoV-2).

### BRIEF DESCRIPTION OF THE DRAWINGS

FIG. 1 shows an overview of the structures of cucurbit[7]uril (CB[7]) and cucurbit[8]uril (CB[8]), as well as the proposed complexing with a platinum centered terpyridine (tpy) therein.

FIG. 2 shows formation of CB[n]-bound Pt/Peptide assemblies. ¹H and ¹⁹F NMR spectra of (a) free peptide FGGGC, as well as assemblies (b) CB[8]^{Pt·}1₂, (c) 1·CB[7]^{Phe} and (d) CB[7]^{Pt}·1·CB[7]^{Phe} in D₂O. Signals pertaining to the amino acids are colored according to the cartoon representation. All chemical shifts in this Figure and in the following ones are in ppm.

FIG. 3 shows recognition of CB[7] by assembly CB[8]^{Pt}·1. ¹H and ¹⁹F NMR spectra of assemblies (a) CB[8]^{Pt}·1₂, and (b) CB[8]^{Pt}·(Pt·1·CB[7])₂; see FIG. 2 for hydrogen labeling. Ticks 0.1 ppm apart on the ¹⁹F NMR spectra chemical shift axis.

FIG. 4 shows CB[7] and CB[8] switching stations upon addition of CB[8]-bound peptide dimer (FGGGC)₂·CB[8] to the CB[7]-bound Pt chloride precursor. Binding affinities obtained by isothermal titration calorimetry.

FIG. 5 shows three scenarios (1-3) considered for the recognition of CB[8] by Pt dimer CB[8]^{Pt}·1₂. ¹H and ¹⁹F NMR spectra of assembly CB[8]^{Pt}·1₂ in D₂O (a) in the absence of CB[8], and in the presence of (b) 0.50 equiv and (c) 1.0 equiv CB[8] relative to assembly CB[8]^{Pt}·1₂; see FIG. 2 for signal labeling. Close-up of spectra (a) and (c) in the 5.3-6.0 ppm region. Dashed signals highlight the signal overlap with CB[8]^{Pt} hydrogens. Ticks 0.1 ppm apart on the ¹⁹F NMR spectra chemical shift axis.

FIG. 6 shows (a) Most stable structure of pendant necklace CB[8]^{Pt}·1₂·CB[8]^{Phe}_{HT} optimized at the B97-3c/def2-mTZVP level. Hydrogen atoms interacting through space are highlighted with white spheres. (b) ¹H-¹H NOESY spectrum of assembly CB[8]^{Pt}·1₂·CB[7]^{Phe}_{HT} where cross-peaks of hydrogens H⁶, H^{a} and H^{b} with CB[8]^{Phe} hydrogens are observed.

FIG. 7 shows opening of pendant necklace CB[8]^{Pt}·(Pt·1)₂·CB[8]^{Phe}_{HT} in the presence of an excess amount of CB[8]. 1H and 19F NMR spectra of (a) peptide 1, as well as assemblies (b) CB[8]^{Pt}·(Pt·1)₂, (c) 1·CB[8], and (d) CB[8]^{Pt}·(Pt·1·CB[8]^{Phe})₂ in the presence of 3.0 equiv CB[8] (mixed with pendant necklace CB[8]^{Pt}·(Pt·1)₂·CB[8]^{Phe}_{HT}). The dashed rectangle highlights the similarity between spectra c and d. Ticks 0.1 ppm apart in the ¹⁹F spectra chemical shift axis.

FIG. 8 shows (a) Docking of a CB[8]-secured Platinum dimer into the active site of the SARS-CoV-2 cysteine protease Mpro (spheres are Platinum centers). (b) a CB[8]-secured Platinum dimer interacting with a short peptide containing both cysteine and histidine residues, as mimic of a larger enzyme. (c) Enzyme Mpro, with the cysteine and histidine pair of the active site. (d) TEVp, a commercially available enzyme that will be used as close mimic of Mpro in laboratory work. (e) Functionalizing of a CB[8]-secured Pt dimer with a peptide tag to enhance its selectivity towards a particular cysteine protease (Mpro or PLpro, for example).

FIGS. 9A-9C shows CB[8]-secured Platinum dimers with simple thiols (FIG. 9A), short peptides bearing one Pt-binding cysteine (FIG. 9B), and longer peptides, such as oxytocin bearing a pair of cysteine residues (FIG. 9C).

FIG. 10 shows the formation of different CB[8] complexes through changes to the respective equivalents of CB[8] added.

### DETAILED DESCRIPTION

### Cysteine Peptide Complexing

The present disclosure concerns cucurbit[n]uril complexes with one or more peptides. Cucurbit[n]urils (CB[n]s or cucurbiturils (CBs)) refer to a family of hollow, pumpkin-shaped macrocycles that can encapsulate various hosts in aqueous medium with extreme affinity. In some aspect, the CB[n]s include macromolecules of glycoluril monomers linked by methylene bridges. A general formula for a CB[n] may be expressed as follows:

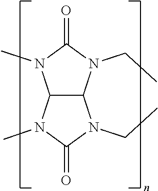

In the general structure for a CB[n], the subscript n, referring to the number of glycoluril monomers, may be an integer from 5 to about 20 or from 5 to about 10, although numbers greater than 20 are contemplated herein as well It should be understood that the general formula of the CB[n] expresses a chain of glycoluril monomers having a first monomer, a last monomer, and a number (n−2) monomers between the first monomer and the last monomer, where the first monomer is linked to the last monomer by the methylene linkage to result in the pumpkin-shaped macrocycle. The greater the integer for n, the larger the diameter of the CB. Thus, in the present disclosure, CB[n] macromolecules formed from the indicated glycoluril monomers are represented by the following shorthand notation to express the three-dimensional arrangement of monomers:

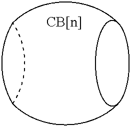

In one aspect of the present disclosure, it has been identified that cucurbit[7]uril and/or cucurbit[8]uril can house or retain or secure one or more peptides. Cucurbit[8]uril (CB[8]) and cucurbit[7]uril (CB[7]) typically form ternary complexes in aqueous medium with pairs of charged guests by distributing the positive charges over both portals of the macrocycle, in a head-to-tail (HT) arrangement. Platinum terpyridyl (tpy) complexes bearing a CB[8]-binding unit at the tpy 4′-position may assemble with CB[8] into a head-to-head (HH) motif, with both positive platinum centers configured on top of each other at one CB[8] portal, leaving the other void of any guest interaction. Favorable dispersive interactions between the stacked tpy ligands and possible metal-metal bonding through d_{z}2-d_{z}2 orbital overlap were proposed as driving forces for the recognition pattern. A variety of thiolates, including cysteine and L-glutathione, were shown that they can be grafted in situ to CB[8]-secured platinum chloride dimers to form large dynamic libraries of homo- and heteroternary assemblies. For example, as both ligand and supramolecular exchanges operate (i.e. the self-pairing of different platinum complexes in the presence of CB[8] in the latter case), a pair of Pt-tpy complexes combined with a pair of thiolate ligands can afford a dynamic pool of up to 10 ternary CB[8]-secured assemblies.

Curcurbituril peptide complexes according to this disclosure include three primary components: (1) at least one platinum-terpyridine-peptide complex; and (2) at least one CB[n] circumposed about a portion of the at least one platinum-terpyridine-peptide complex, such that the portion of the at least one platinum-terpyridine-peptide complex is disposed and fixed within the internal cavity of the CB[n] macrocycle.

The first primary component of the curcurbituril peptide complexes is a platinum-terpyridine-peptide complex according to formula (I):

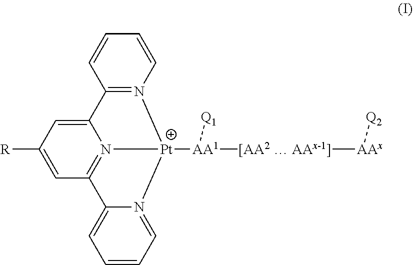

The platinum-terpyridine-peptide complex according to formula (I) includes a functional group R on the terpyridine ligand coordinated with a platinum metal center. A peptide is bound to the platinum metal center opposite the terpyridine ligand. It should be understood that as used herein, “peptide” refers to at least two amino acids linked via a peptide bond and may include polypeptides and proteins. In some aspects, the peptide includes a cysteine and/or histidine amino acid within its sequence. In some aspects, the peptide includes a cysteine or histidine at at least one terminus or within two or three amino acids thereof. In some aspects, the peptide is bound to the platinum metal center through a platinum-sulfur bond or a platinum-nitrogen bond.

The peptide of the platinum-terpyridine-peptide complex according to formula (I) is composed of ‘x’ amino acids (AA), where AA¹ is a bound amino acid, in the sense that it is directly bound to the platinum metal center, AA^{X} is a terminal or distal amino acid of the peptide, and the other amino acids [AA² . . . AA^{X-1}] are intermediate amino acids in the peptide. The bound amino acid AA¹ may be at a terminus of the peptide, but need not necessarily be at an end of the peptide. Similarly, AA^{X} may be a terminus of the peptide, such as the opposite the terminal amino acid to AA¹ The bound amino acid AA¹ may be in the middle of the peptide, as indicated by the optional group Q₁ and Q₂ in formula (I), where Q₁ and Q₂ are independently either non-existant or one or more additional amino acids not between the bound amino acid AA¹ and the terminal or distal amino acid AA^{X}. The peptide has at least two amino acids.

In some aspects, the peptide is from 2 to 5000 amino acids in length. In specific examples, the peptide is from 2 to 50, from 2 to 20, from 2 to 10, from 2 to 5, from 5 to 50, from 5 to 20, or from 5 to 10 amino acids in length. In some aspects, the peptide is of about 250 Daltons (Da) or more, including about 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, or more Da. In some aspects, the peptide may be of about 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500 or more kDa.

In some aspects, the peptide includes a thiol or an imidazole at at least one terminus. In some aspects, the thiol is a cysteine residue. In some aspects, the imidazole is a histidine residue. In some aspects, a cysteine residue and/or histidine is located at at least the carboxyl terminus of the peptide. In some aspects, the cysteine or histidine residue at the carboxyl terminus conjugates or binds the peptide to the Pt-tpy to hold the peptide within the curcurbituril complex. In further aspects, a cysteine and/or histidine at a position internal to the peptide or protein sequence conjugates to the platinum of the Pt-tpy.

The functional group R of the platinum-terpyridine-peptide complex according to formula (I) may be phenyl or substituted phenyl such as, for example, a methylphenyl (tolyl), a phenol, or a halophenyl. Examples of methylphenyl groups R include 3-methylphenyl, 4-methylphenyl, and 5-methylphenyl. Examples of halophenyl groups R include chlorophenyl, dichlorophenyl, fluorophenyl, and difluorophenyl. In specific examples, R may be 3-chlorophenyl, 4-chlorophenyl, 5-chlorophenyl, 3-fluorophenyl, 4-fluorophenyl, 5-fluorophenyl, 3,5-dichlorophenyl, or 3,5-difluorophenyl. In some aspects, the platinum-terpyridine portion of the platinum-terpyridine-peptide complex is 4′-(3,5-difluorophenyl)-2,2′:6′,2″-terpyridine platinum (II) or 4′-(4-methylphenyl)-2,2′:6′,2″-terpyridine platinum (II). In some aspects, the peptide is conjugated to the platinum center through a thiol or imidazole group in the peptide, such as a cysteine and/or a selenocysteine and/or a histidine or via an attached glutathione. In some aspects, the thiol or imidazole is located at or near the amino and/or carboxyl terminus.

To facilitate further description of the curcurbituril peptide complexes, the functional group R of the platinum-terpyridine-peptide complex is designated herein as the “head portion” of the platinum-terpyridine-peptide complex. Further, the terminal amino acid AA^{X} of the platinum-terpyridine-peptide complex is designated as the “tail portion” of the platinum-terpyridine-peptide complex.

The second primary component of the curcurbituril peptide complexes is at least one CB[n] circumposed about a portion of the platinum-terpyridine-peptide complex, such that the portion of the platinum-terpyridine-peptide complex is disposed in, or fixed or secured within, the internal cavity of the CB[n] macrocycle. The circumposed portion may be a head portion or a tail portion of the platinum-terpyridine-peptide complex. In some examples, the CB[n] may be CB[7] or CB[8]. Further examples are contemplated, in which the CB[n] is CB[9] or CB[10].

In one example of the curcurbituril peptide complex, the at least one CB[n] is circumposed about the head portion of the platinum-terpyridine-peptide complex. Thus, in some aspects, the curcurbituril peptide complex is a complex according to Formula (II):

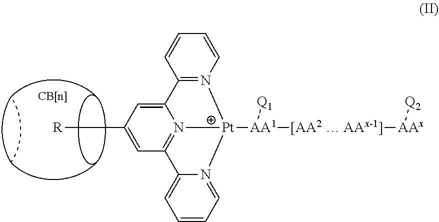

In formula (II), CB[n], R, AA, Q₁ and Q₂ are as defined in formula (I).

In some aspects, the CB secures or hosts the Pt-tpy to form a complex. In some aspects, the CB hosts or secures the “guest” Pt-tpy through affinity for positive charges or cations, possibly through carbonyl groups lining the cavity. In some aspects, the CB secures the Pt-tpy through interaction with at least the R group of Formula (I), as depicted in Formula (II). In some aspects, the Pt-tpy can be pre-conjugated to a peptide. In other aspects, the Pt-tpy can be utilized to bind or capture desired peptides, such as be administration to a cell or cell lysate or incubation with proteinacious material. In some aspects, a halogen, such as a chloride, an iodide, a bromide, or a fluoride, may be utilized to neutralize the platinum charge, wherein the halogen will readily depart and allow the peptide to conjugate or bind thereto. In some aspects, a pre-conjugated peptide can also be used to bind or capture proteins via incubation based on protein-protein interactions. In some aspects, the peptide includes a terminal cysteine or histidine, a terminal phenylalanine, and at least one neutral amino acid therebetween, such as at least one glycine.

In another example of the curcurbituril peptide complex, the curcurbituril peptide complex includes two CB[n], where a first CB[n] is circumposed about the head portion of the platinum-terpyridine-peptide complex and a second CB[n] is circumposed about the tail portion of the platinum-terpyridine-peptide complex. Thus, in some aspects, the curcurbituril peptide complex is a complex according to Formula (III):

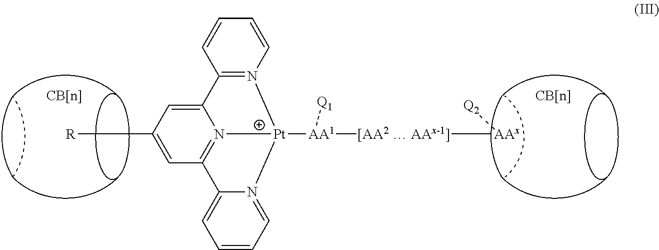

In formula (III), each CB[n], R, AA, Q₁ and Q₂ are as defined in formula (I).

As identified herein, the presence of a phenyl or appended phenyl can allow for a CB to interact and enshroud the residue. As such, the presence of a free phenyl, such as with phenylalanine or tyrosine allows for other CBs to complex therewith. It will be appreciated that in addition to side chains of amino acids, additional steps can be performed to append a desired group on or near the terminus of a peptide.

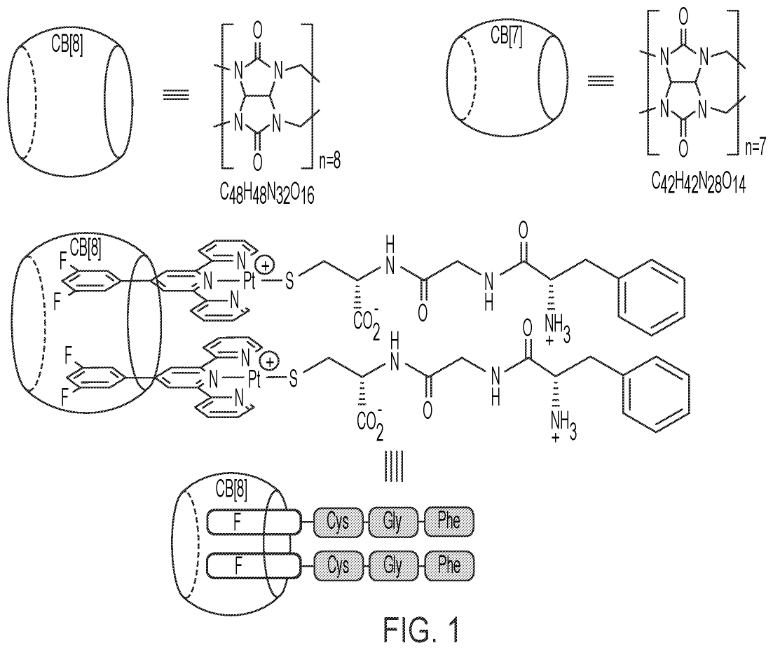

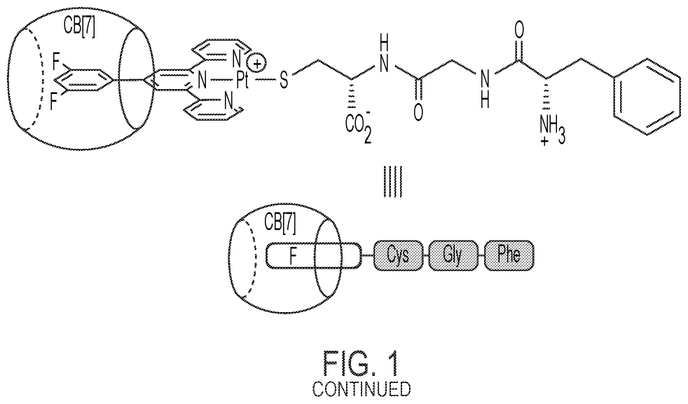

In another example of the curcurbituril peptide complex, as illustrated at the top of FIG. 1, the curcurbituril peptide complex includes two platinum-terpyridine-peptide complexes and one CB[n], in which the CB[n] is circumposed about both head portions of the two platinum-terpyridine-peptide complexes. In this example, the CB[n] is a CB[8].

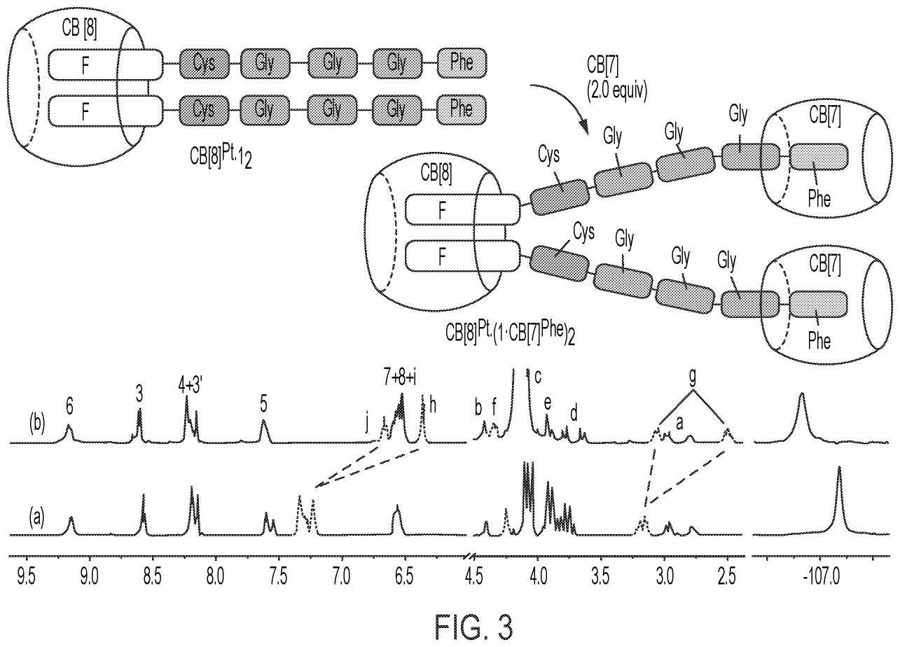

In another example of the curcurbituril peptide complex, as illustrated in FIG. 3, the curcurbituril peptide complex includes two platinum-terpyridine-peptide complexes and three CB[n], in which a first CB[n] is circumposed about both head portions of the two platinum-terpyridine-peptide complexes, a second CB[n] is circumposed about the tail portion of one of a first of the two platinum-terpyridine-peptide complexes, and a third CB[n] is circumposed about the tail portion of a second of the two platinum-terpyridine-peptide complexes. In this example, the first CB[n] is a CB[8], the second CB[n] is a CB[7], and the third CB[n] is a CB[7].

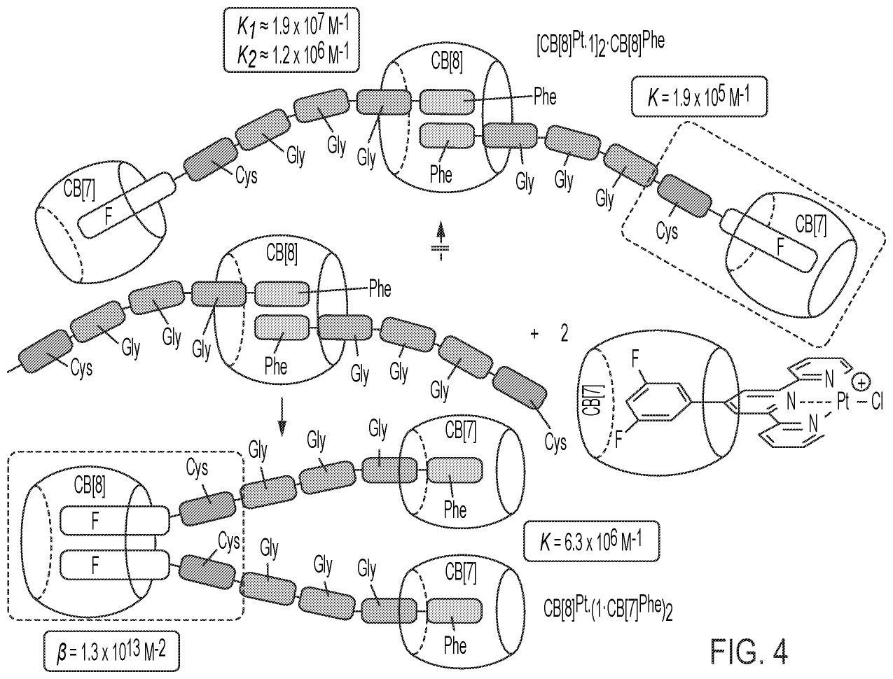

In another example of the curcurbituril peptide complex, as illustrated at the top of FIG. 4, the curcurbituril peptide complex includes two platinum-terpyridine-peptide complexes and three CB[n], in which a first CB[n] is circumposed about the head portion of a first of the two platinum-terpyridine-peptide complexes, a second CB[n] is circumposed about the head portion of a second of the two platinum-terpyridine-peptide complexes, and a third CB[n] is circumposed about the two tail portions of both two platinum-terpyridine-peptide complexes. In this example, the first CB[n] is a CB[7], the second CB[n] is a CB[7], and the third CB[n] is a CB[8].

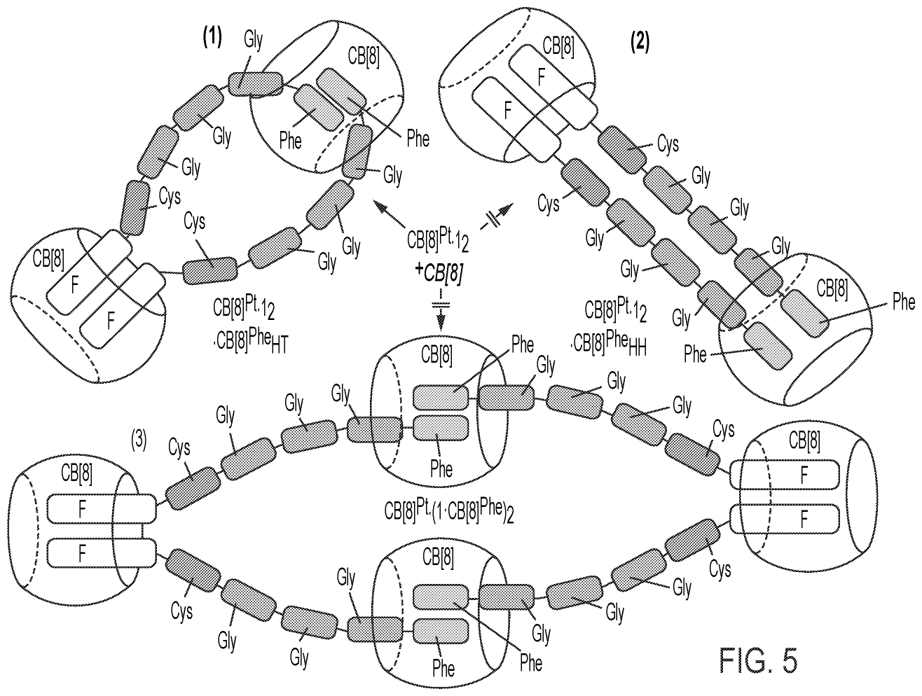

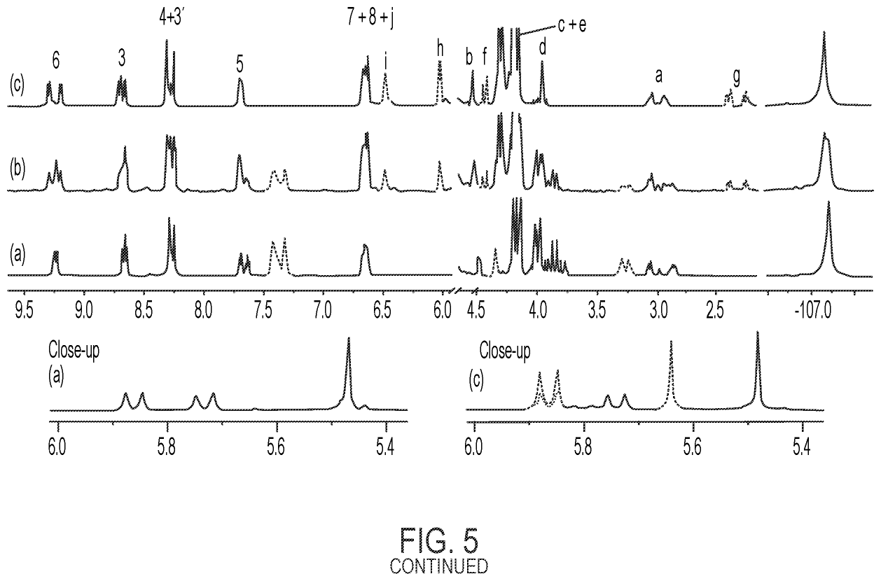

In another example of the curcurbituril peptide complex, as illustrated as complexes (1) and (2) of FIG. 5, the curcurbituril peptide complex includes two platinum-terpyridine-peptide complexes and two CB[n], in which a first CB[n] is circumposed about the head portions of the two platinum-terpyridine-peptide complexes, and a second CB[n] is circumposed about the two tail portions of both two platinum-terpyridine-peptide complexes. In this example, the first CB[n] is a CB[8], and the second CB[n] is a CB[8]. The complexes (1) and (2) are distinguished from each other, based on the orientations of the tail portions. In complex (1), the two tail portions enter the CB[8] from opposite ends of the CB[8], whereas in complex (2), the two tail portions enter the CB[8] from the same end of the CB[8].

In another example of the curcurbituril peptide complex, as illustrated as complex (3) of FIG. 5, the curcurbituril peptide complex includes four platinum-terpyridine-peptide complexes and four CB[n]. In complex (3), a first CB[n] is circumposed about the head portions of a first and second of the four platinum-terpyridine-peptide complexes, and a second CB[n] is circumposed about the head portions of a third and fourth of the four platinum-terpyridine-peptide complexes. A third CB[n] is circumposed about the two tail portions of the first and third platinum-terpyridine-peptide complexes, and a fourth CB[n] is circumposed about the two tail portions of the second and fourth platinum-terpyridine-peptide complexes. In this example, the first CB[n] is a CB[8], the second CB[n] is a CB[8], the third CB[n] is a CB[8], and the fourth CB[n] is a CB[8].

In some aspects, the CB complex of CB[7] or CB[8] coupled with a Pt-tpy can be used to bind proteins through one or more cysteine and/or histidine residues therein. As identified herein, the platinum is positively charged or at least positively charged in solution or following de-halogenation within the complex and will accordingly react favorably with the vulnerable sulfur group of the cysteine side-chain or imidazole group of the histidine. It will be appreciated that the cysteine and/or histidine need not be located at or towards the terminus of the protein or peptide, as the binding is through the side-chain of the amino acid.

In some aspects, the present disclosure also concerns forming CB complexes with multiple CB units therein. In addition to CBs retaining the Pt-tpy units, CBs will also retain phenylalanine (Phe) or tyrosine (Tyr) through the phenyl on the amino acid's side-chain. As is depicted herein, CB[8] will retain up to two Pt-tpy units, two Phe residues, two Tyr residues, or a combination thereof and CB[7] will retain only one Phe/Tyr residue or one Pt-tpy. It is therefore a further aspect of the present disclosure to provide more elaborate CB-complexes of two or more CB units. In some aspects, a terminus of the peptide is housed within a further CB unit, such as a CB[7] or CB[8]. It will be appreciated that the CB[8] may be circumposed about a further peptide terminus or intermediate amino acid, either in an HH or HT configuration. In some aspects, a CB complex may include a CB[8] with two Pt-tpy-peptide complexes, wherein the opposing termini are housed in one or more CB[8]s and/or CB[7]s. By way of example, FIGS. 4 and 5 set forth several possible configurations. As set forth herein, providing varying equivalents of the CBs to peptide concentration allows for the preferred complex to be formed.

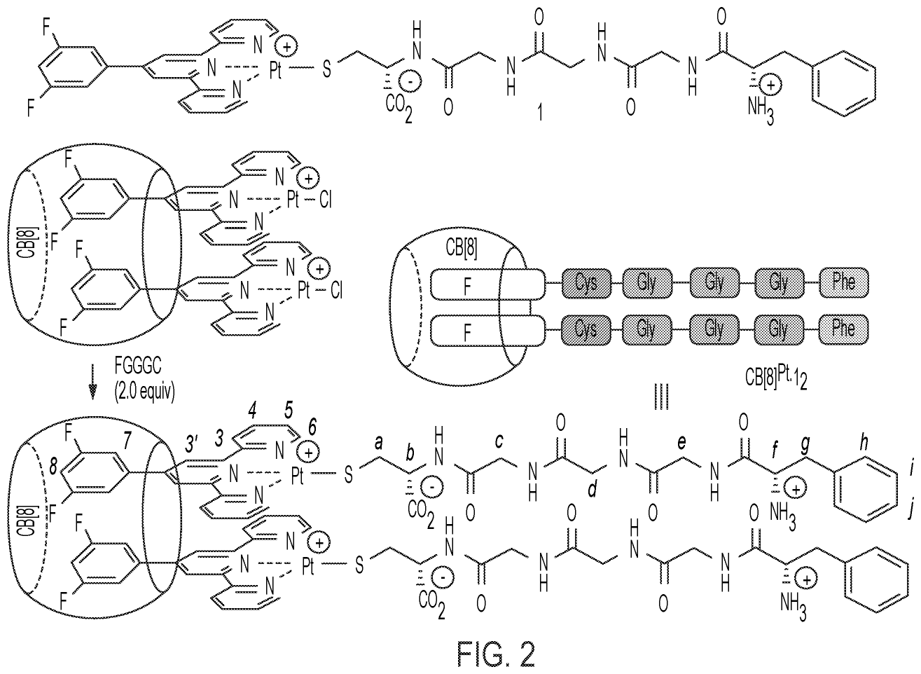

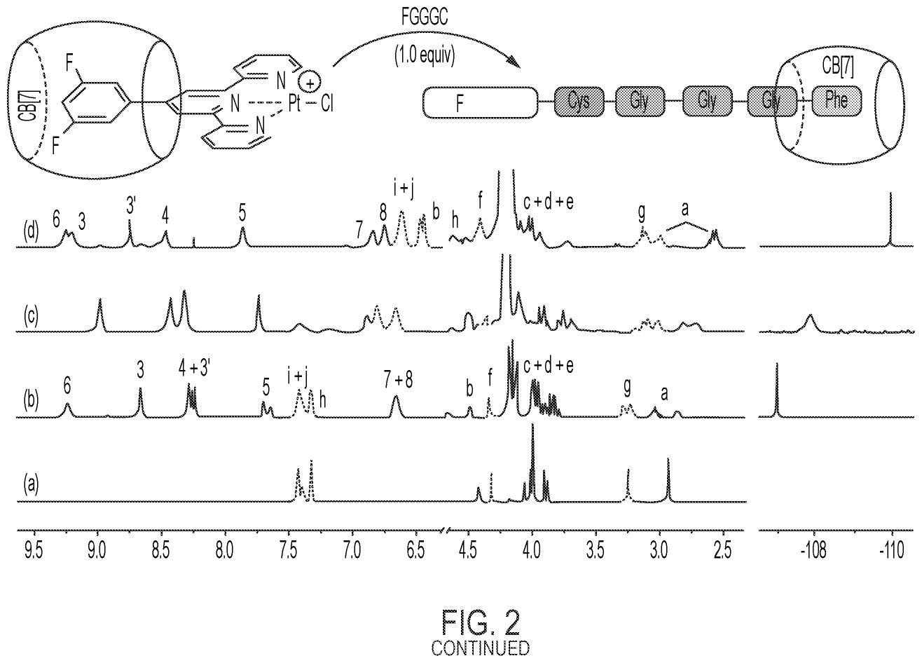

In some aspects, the present disclosure concerns one initial step was to test whether it was possible to (1) form well-defined head-to-head (HH) CB[8]-secured Pt/peptide dimers, and (2) use those assemblies to target a subsequent host, again in a well-defined manner. The Examples herein demonstrate that both can be achieved. Using a pentapeptide Phe-(Gly)₃-Cys (FGGGC; 1) and either CB[7] or CB[8] as secondary hosts to secure the peptide via the Pt-tpy. The present disclosure also demonstrates that elegant structures such as necklaces with both HH and head-to-tail (HT) features can be obtained in situ from just a few readily available building blocks in aqueous medium. Pentapeptide FGGGC was chosen as terminal phenylalanines (Phe) will form tight binary complexes with CB[7] (binding affinity up to 1.8×10⁶ M⁻¹), and strong HT ternary assemblies with CB[8] (binding affinity up to 1.1×10⁸ M⁻²; (see Examples below for the titration of pentapeptide 1 with CB[8]). The cysteine residue binds to the Pt center(s) and the three glycines were introduced to act as non-reactive spacers that confer flexibility to the side chain. For the sake of clarity, “Pt” or “Phe” superscripts are added to CB[n] to indicate which portion of the Pt/peptide complex the macrocycles interact with. As control experiments, the behavior of the CB[8]-secured Pt dimers was compared with CB[7]-bound Pt monomers (see FIG. 2).

Pt/peptide assembly CB[8]^{Pt}·1₂ (see FIG. 2) was readily obtained in situ by substitution of the chloride ligand from the parent Pt chloride assembly with pentapeptide FGGGC in deuterium oxide. No buffer was used as the charge of Pt/peptide complex 1 should remain singly positive over a wide pH (or pD) range (approximately 2-9). Upon dimer formation at the Pt tpy site, terpyridyl hydrogens H⁶ are shifted downfield by 0.33 ppm and split into two doublets, as they become diastereotopic in the presence of the two chiral peptides (see FIG. 2, spectrum b). This behavior was also observed for aromatic hydrogens at positions 3-5 and 3′, as was expected. Signals pertaining to peptide FGGGC are barely affected except for diastereotopic hydrogens H^{a}, which split into two multiplets separated by 0.08 ppm. On the other hand, fluorine atoms located at the tpy head were observed to experience an upfield shift (0.19 ppm) upon peptide binding.

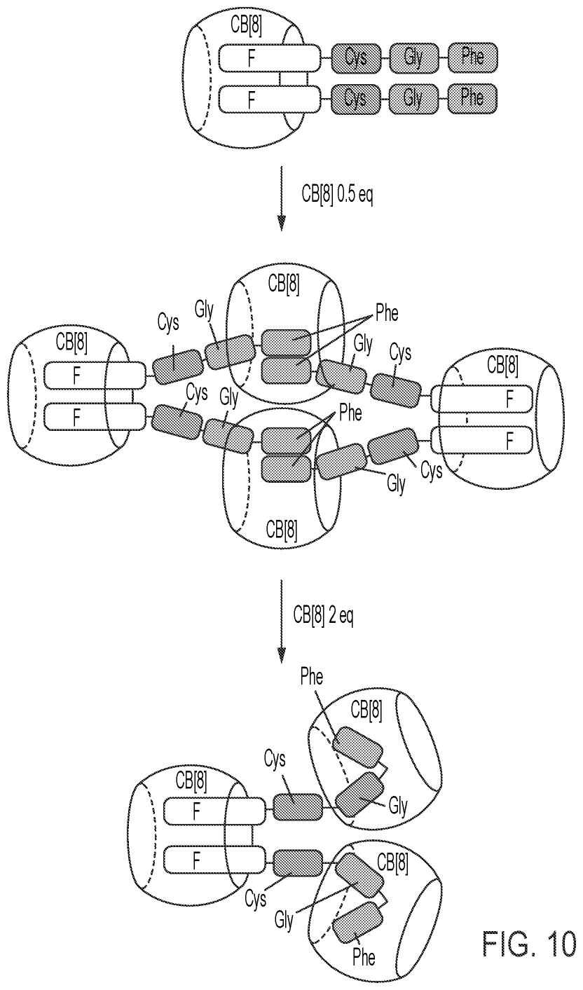

Treatment of the CB[7]-bound Pt chloride assembly with the pentapeptide, however, afforded mostly assembly 1·CB[7]^{Phe}, with CB[7] switching from the Pt tpy to the Phe station (see FIG. 2, spectrum (c)). In the presence of an excess amount of CB[7] (>2 equiv), both units were encapsulated by the macrocycle (see FIG. 10, spectrum d).

The CB[8]^{Pt}·1₂ assembly was then titrated with CB[7] to test the stability of the CB[8]-secured Pt dimer in the presence of a competing target host. [5]Pseudorotaxane CB[8]^{Pt}·(1·CB[7]^{Phe})₂ was formed exclusively after addition of 2.0 equiv CB[7] (see FIG. 3). No association between the tpy head and CB[7] was observed, i.e. no disassembly of the CB[8]-secured dimer seemed to take place. Significant upfield shifts were observed for aromatic hydrogens H^{h}, Hⁱ and H^{j} (0.64-0.88 ppm) and methylene hydrogens H^{g} (0.64 ppm), and downfield shifts for hydrogens H^{f} (0.10 ppm). Those shifts confirm the inclusion of the benzyl moiety within CB[7], with the α-carbonyl hydrogen H^{f} located near the rim of the macrocycle. As expected, no significant shifts were observed for the remote fluorine nuclei on the tpy heads.

Remarkably, [5]Pseudorotaxane CB[8]^{Pt}·(1·CB[7]^{Phe})₂ is also formed exclusively when ternary assembly (FGGGC)₂·CB[8] is treated with 2.0 equiv. of the CB[7]-bound Pt tpy chloride precursor (see FIG. 3). The expected [CB[7]^{Pt}·1]₂·CB[8]^{Phe} assembly was not detected, even immediately after mixing.

To quantify the CB[8] preference for the Pt tpy sites over the Phe residues, the various recognition events at play were analyzed by isothermal titration calorimetry (ITC). The affinities of CB[7] towards the Phe residue of pentapeptide FGGGC and the CB[8]^{Pt}·1₂ complex are 1.2 (±0.2)′10⁷ M⁻¹ and 6.3 (±0.2)′10⁶ M⁻¹ (see FIG. 4), respectively, in excellent agreement with the binding affinities measured by Urbach for the FGG peptide (2.8′10⁶ M⁻¹ in a 10 mM sodium phosphate buffer, pH 7.0) (see, Urbach et al., Isr. J. Chem., 2011, 51: 664-678; Logsdon et al., J. Am. Chem. Soc., 2011, 133: 17087-17092).

The proximity of both peptide chains in complex CB[8]^{Pt}·1₂ thus does not cause any cooperativity effect, i.e. grafting the peptide to the CB[8]-secured Pt dimer scaffold does not significantly impact the binding affinity of the terminal Phe unit. To determine the affinity of CB[7] towards the difluoroaryl substituent of the tpy ligand without perturbation from the Phe binding site, titrations were carried out using the truncated CB[8]-secured Pt/cysteine complex as shown in FIG. 4. The affinity of this complex towards CB[7] is only 1.9 (±0.1)′10⁵ M⁻¹. The affinities of pentapeptide FGGGC towards CB[8] were 1.9 (±0.6)′10⁷ M⁻¹ and 1.2 (±0.6)′10⁶ M⁻¹ for the formation of the binary and HT ternary complexes, respectively, again in excellent agreement with reported binding affinities of N-terminal Phe residues in short peptides and proteins. Cooperativity is quantified using equation 1, where K₁ and K₂ are the equilibrium constants for the formation of binary and ternary complexes, and a is an interaction parameter; positive and negative cooperativities are observed when a>1 and a<1, respectively. In our case, cooperativity in the CB[8] encapsulation of the pair of Phe residues is slightly negative (a=0.26±0.15).

$$α = \frac{4 {K}_{2}}{{K}_{1}} \qquad ( 1 )$$

Caution may be warranted, however, for as shown by Urbach and coworkers (J. Am. Chem. Soc. 2006, 128, 12574-12581) in the case of tripeptide FGG and CB[8], as well as Cistola and coworkers (Proc. Natl. Acad. Sci. USA 2002, 99, 1847-1852) with small molecule/protein interactions, K₁ and K₂ constants obtained by ITC can be strongly correlated, i.e. (1) reasonable fits of ITC enthalpograms can be obtained when setting K₁ as a constant while fitting K₂; and (2) ternary binding constant b=K₁K₂ (in M⁻²) is rather insensitive to the value of K₁. In the present disclosure, a plot of the goodness-of-fit value c² as a function of K₁ returns a clear minimum at the K₁ constant mentioned above. Furthermore, the error on parameter a is small enough to ascertain that negative cooperativity is much more likely than not.

Again the truncated Pt-tpy-cysteine complex was used to determine the affinity of CB[8] towards the difluoroaryl unit of the tpy ligand (FIG. 4). Equilibria (2) and (3) were used to fit the enthalpogram, returning equilibrium constants Kₚₜ₋ₚₜ and b equal to 2.0 (±1.1)×10⁴ M⁻¹ and 1.3 (±0.6)×10¹³ M⁻², respectively.

Pt·Cys+Pt·Cys⇄(Pt·Cys)₂ (2)

2Pt·Cys+CB[8]⇄CB[8]^{Phe}·(Pt·Cys)₂ (3)

The dimerization constant of the Pt/Cys assembly K_{Pt-Pt} corresponds to a free energy term of −5.9 (±0.3) kcal/mol, in excellent agreement with the typical strength of Pt-Pt interactions. It is noted that this dimerization in the absence of CB[8] prevents from extracting separate binding constants K₁ and K₂ towards the macrocycle. A reliable ternary binding constant b=K₁K₂ was obtained, however (1.3 (±0.6)×10¹³ M⁻²). In other terms, the Pt-tpy-Cys dimer can be considered as a standalone guest forming a 1:1 complex with CB[8], with a binding affinity K′ of 7 (±5)×10⁸ M⁻¹, obtained from equation (4).

$${K}^{′} = \frac{β}{{K}_{P t - P t}} \qquad ( 4 )$$

The combined equilibrium constant for the formation of assemblies [CB[7]^{Pt}·1]₂·CB[8]^{Phe} (FIG. 4) and CB[8]^{Pt}·(1·CB[7]^{Phe})₂ (lower section of FIG. 3) from the free hosts and guests are thus 8.1′10²³ and 5.2′10²⁶ M⁻⁴, respectively. This 640-fold difference corresponds to a 3.8 kcal/mol preference for assembly CB[8]^{Pt}·(1·CB[7]^{Phe})₂, which supports its exclusive formation.

The recognition of CB[8]-secured Pt dimer CB[8]^{Pt}·1₂ towards CB[8] as the target host was subsequently tested. In the presence of 0.5 equiv of the macrocycle (relative to Pt), i.e. 1.0 equiv relative to assembly CB[8]^{Pt}·1₂, at least three well-defined assemblies are plausible (see FIG. 5): (1) a supramolecular “pendant necklace” with a 2:2 Pt-peptide/CB[8] stoichiometry in hybrid HH and HT arrangements at the tpy units and Phe residues, respectively (CB[8]^{Pt}·1₂·CB[8]^{Phe}_{HT}), (2) a structure with the same stoichiometry but in a double HH arrangement (CB[8]^{Pt}·1₂·CB[8]^{Phe}_{HH}), and (3) a HT dimer of assemblies with a 4:4 stoichiometry [CB[8]^{Pt}·1₂·CB[8]^{Phe}]₂.

New ¹H NMR signals appeared immediately after adding a first CB[8] aliquot (0.50 equiv; see FIG. 5, spectrum b), showing the formation of a new assembly in a slow exchange regime. A very well-defined set of new signals is present after addition of exactly 1.0 equiv CB[8] relative to assembly CB[8]^{Pt}·1₂ (FIG. 5, spectrum c). This led to the suspicion that only one of the assemblies outlined above is present in the mixture. The formation of a new, unique ¹⁹F NMR signal at 1.0 equiv CB[8] (−107.06 ppm) is consistent with the observations made from the ¹H NMR spectra.

A comparison of spectra a and c in FIG. 5 shows a large upheld shift for hydrogens H^{g}, H^{h}, Hⁱ and H^{j} (0.90, 1.29, 0.79 and 0.90 ppm, respectively), a very similar behavior already observed for aromatic hydrogens in the CB[8]-bound peptide dimer FGGGC₂·CB[8]. This clearly indicates that the recognition event is taking place at the Phe residue. The 5.3-6.0 ppm portion of the ¹H NMR spectrum displays a feature that also allows us to discard assembly CB[8]^{Pt}·1₂·CB[8]^{Phe}_{HH} with the double HH arrangement (see FIG. 5, scenario 2): the CB[8] rims in such an assembly would all be non-equivalent, and would afford 2 pairs of doublets for the pseudo-equatorial hydrogens. This is not the case; addition of 0.50 equiv CB[8] to assembly CB[8]^{Pt}·1₂ shows the growth of only one doublet at 5.85 ppm (see FIG. 5, in spectrum c close-up), in addition to the expected signal of the new CB[8]^{Phe} equatorial hydrogens (5.65 ppm). This is evidence that the newly bound CB[8]^{Phe} has equivalent portals.

To discriminate between assembly CB[8]^{Pt}·1₂·CB[8]^{Phe}_{HT} and dimer [CB[8]^{Pt}·1₂·CB[8]^{Phe}]₂ (see FIG. 5, scenarios 1 and 3), the diffusion coefficients of the unknown assembly were compared with that of complex CB[8]^{Pt}·1₂ using diffusion-ordered spectroscopy (DOSY) experiments. Diffusion constants in D₂O were 1.58 (±0.04)×10⁻¹⁰ m²s⁻¹ and 1.51 (±0.01)−10⁻¹⁰ m²s⁻¹, respectively. A difference as low as 7 (±5)×10⁻¹² m²s⁻¹ indicates that the hydrodynamic radius of the unknown assembly is very similar to CB[8]^{Pt}·1₂. Assembly CB[8]^{Pt}·1₂·CB[8]^{Phe}_{HT}, with hybrid HH and HT arrangements at the tpy ligand and Phe residues, respectively (see FIG. 5, scenario 1) thus becomes the only plausible option. It is preferred over the entropically penalizing 4:4 assembly [CB[8]^{Pt}·1₂·CB[8]^{Phe}]₂. Mass spectrometry analysis further confirmed the formation of the shorter “pendant necklace.”

Binding constants for the formation of the binary and ternary complexes at the Phe residue were K₁=2.4 (±0.3)′10⁶ M⁻¹ and K₂=1.7 (±0.4)′10⁶ M⁻¹, respectively. Again, a plot of the goodness-of-fit value c² as a function of K₁ returns a clear minimum at the K₁ constant mentioned above. While attempts to rationalize the mild differences with the CB[8] encapsulation of free pentapeptide FGGGC would be putative, two trends deserve mentioning: (1) positive cooperativity is now observed (a=2.8±0.7) while cooperativity is negative in the case of free peptide FGGGC (a=0.26±0.15); and (2) the formation of the “pendant necklace” (i.e. the formation of the ternary complex at the Phe residues) is entropically neutral (TDS₂=−0.1 kcal/mol), while ternary complex formation with the free peptide is entropically penalizing (TDS₂=−2.0 kcal/mol). Both trends suggest that the entropically favorable “intra-assembly” necklace formation overcompensates the concomitant, entropically-penalizing restriction of conformational mobility.

It is further noted here that the term “pendant necklace” is used for the lack of a better word to qualify structure CB[8]^{Pt}·1₂·CB[8]^{Phe}_{HT}. Topologically, it is a [2]pseudocatenane, with CB[8]^{Phe} being one ring component and the chain 1·CB[8]^{Pt}·1 the other ring component. However, this simple nomenclature would consider the chain 1·CB[8]^{Pt}·1 as one unit, without taking into account that it is itself a [3]pseudorotaxane. Ultimately, we propose the term “pendant necklace”, as the CB[8]-secured Pt tpy dimer reminds us of the pendant unit of the jewelry, and the HT ternary complex between the pair of Phe residues and CB[8]^{Phe} its clasp behind the neck.

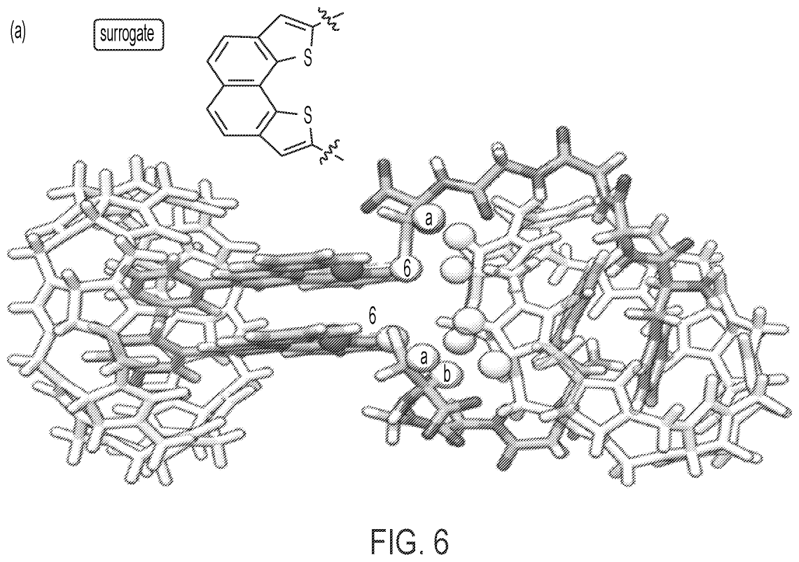

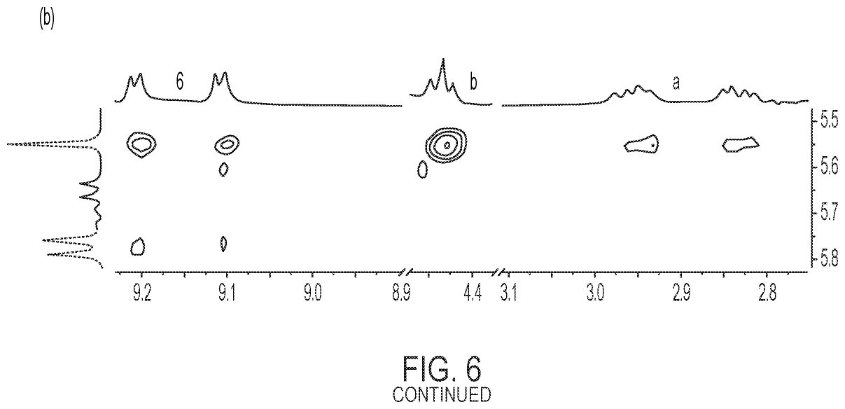

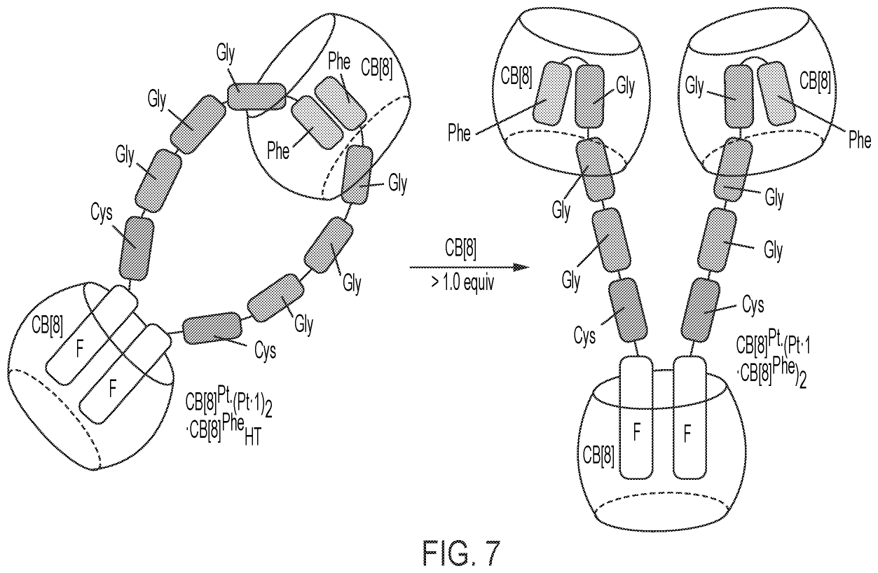

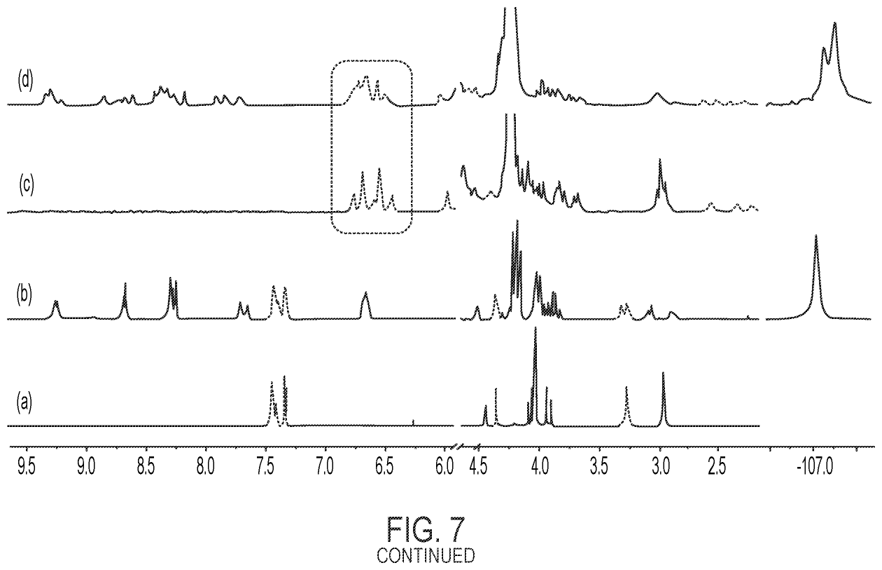

As obtaining crystals of the pendant necklace for X-ray diffraction analysis was unsuccessful, its structure was instead explored using the most up-to-date combination of molecular dynamics, semi-empirical methods and density functional theory being currently developed by Grimme and co-workers (J. Chem. Theory Comput., 2019, 15: 2847-2862; Phys. Chem. Chem. Phys. 2020, 22: 7169-7192; Angew. Chem., Int. Ed. 2020, 59: 15665-15673). Conformational screening was first carried out using an approximate geometrical analog of the pendant necklace bearing only one CB[8] unit (see FIG. 6, part (a)) using Grimme's Conformer-Rotamer Ensemble Sampling Tool (CREST) and the built-in generic GFN-Force-Field (GFN-FF), in conjunction with the GBSA solvation model. The CB[8]-secured Pt dimer surrogate was designed to mimic the distance between the sulfur atoms and the direction of its substituent in the pendant necklace, while increasing computing efficacy. The 37 most stable candidates (out of approximately 12,000; a 25 kcal/mol cut-off was applied) were isolated, and the surrogate fragment was replaced by the CB[8]-secured Pt dimer motif. The structures were then reoptimized with the semiempirical tight-binding method GFN2-xTB. in conjunction with the GBSA solvation model. The four most stable structures (based on a 10 kcal/mol cut-off) were finally reoptimized by DFT using a functional well suited for supramolecular systems (B97-3c; basis sets are def2-mTZVP) in conjunction with the COSMO solvation model. The stability (DG) of the four assemblies was calculated using equation (5), where DE_{B97-3c} is the electronic contribution at 0 K calculated by DFT in the gas phase, DG_{T,xTB} is the vibrational contribution at 25° C. obtained at the GFN2-xTB level, and DG_{solv,xTB} is the solvation energy calculated with the GBSA model at the GFN2-xTB level.

ΔG=ΔE_{B97-3c}+ΔG_{T,xTB}+ΔG_{solv,xTB} (5)

The most stable conformation of the pendant necklace is presented in FIG. 6. The compact arrangement of the pendant necklace suggests that through-space interactions between hydrogens of the peptide and of CB[8] might be observable. A ¹H—¹H NOESY experiment indeed revealed cross-peaks between equatorial hydrogens of CB[8]^{Phe} and tpy hydrogen H⁶, as well as peptidic hydrogens H^{a} and H^{b} (see FIG. 6). Favorable interactions between the peptide carboxylate units and the outer wall of CB[8]^{Phe} might also further compact the assembly.

It is accordingly demonstrated that it is possible to functionalize CB[8]-secured Pt dimers in situ and quantitatively with a pair of cysteine-containing peptides, and use the Pt/peptide/CB[8] assembly to target secondary hosts CB[7] and CB[8] site-selectively. The more elegant outcomes include (1) the formation of pendant necklace CB[8]^{Pt}·1₂·CB[8]^{Phe}_{HT} with a new hydrid HH and HT arrangement at the tpy head and Phe residues, respectively, and a thorough quantification of all recognition forces at play, (2) the successful in silico screening and isolation of a plausible geometry for this necklace, and (3) the quantitative switching of CB[7] and CB[8] from the tpy head and Phe residues, respectively, when attempting to attach the Cys units of ternary complex 1₂·CB[8] to the Pt centers of binary assembly CB[7]^{Pt}·(Pt·Cl). This accordingly paves the way for the recognition of proteins by CB[8]-secured Pt dimers, and for the design of rationally constrained oligopeptides.

It was next addressed whether a shorter peptide might behave similarly. Accordingly a Cys-Gly-Phe peptide was obtained and contacted with the CB[8] and CB[7] Pt-tpy constructs to establish an initial tripeptide complex (see, FIG. 1). By varying the equivalent amounts of CB[8] and/or CB[7] included, different geometries were achieved (see, FIG. 10). Table 1 below sets forth values obtained for different complexes formed through changes in the equivalent amount of CB[8] or CB[7] added. The tripeptide of Cys-Gly-Phe alone forms a HT dimer, but as with the pentapeptide forms a HH dimer when conjugated to a Pt-tpy.

TABLE 1

| Compound | D (m² − s⁻¹) | Log D |
|---|---|---|
| CB[8] CGF-(peptide only dimer) | 2.07 (±0.02)*10⁻¹⁰ | −9.68 |
| CB[8]-diFPtCGF | 1.80 (±0.05)*10⁻¹⁰ | −9.74 |
| CB[8]-diFPtCGF + 0.5 eq CB[8] | 1.13 (±0.02)*10⁻¹⁰ | −9.95 |
| CB[8]-diFPtCGF + 2 eq CB[8] | 1.60 (±0.05)*10⁻¹⁰ | −9.79 |
| CB[8]-diFPtCGF + 2 eq CB[7] | 1.53 (±0.05)*10⁻¹⁰ | −9.82 |
| CB[8]-diFPtCGGGF | 1.58 (±0.04)*10⁻¹⁰ | −9.80 |
| CB[8]-diFPtCGGGF + 0.5 eq CB[8] | 1.51 (±0.03)*10⁻¹⁰ | −9.82 |

diFPt = 4′-(3,5-difluorophenyl)-2,2′:6′,2″-terpyridine platinum (II);
CGF = Cys-Gly-Phe;
CGGGF = Cys-Gly-Gly-Gly-Phe

The present disclosure also concerns methods for making and using the CB complexes as set forth herein. As identified herein, varying the amount or equivalent amount of CB[8] and/or CB[7] to the amount of peptide and/or Pt-tpy and/or Pt-tpy-peptide allows for the formation of the elegant geometries as described herein. In some aspects, the present disclosure further includes contacting or administration of the CB complexes as set forth herein with a cell, cell extract, or cell lysate, including in vitro, in vivo, and ex situ. In some aspects, the CB complex is provided to a virus or to a subject carrying the virus or suspected of carrying the virus.

It will be appreciated that the ability of the CB complex to bind peptides will allow for proteins in vivo and in vitro to complex therewith, either directly to the platinum or secondarily by binding or interacting with a peptide or protein already conjugated to the platinum center of the Pt-tpy. It will also be appreciated that the presence of platinum can provide a toxic effect. By selectively binding particular proteins, the CB complex can selectively target platinum delivery, allowing for a concentrated and selective delivery of the toxic metal. For example, as set forth herein, the selective binding of a viral protease can provide the further effect of selective delivery to the virus, allowing for both a lower dose to achieve a lethal effect by selective concentration, as well as a reduction in unwanted cytotoxicity to the host's own cells.

### CB[8] Pt Dimer Cysteine Protease Complexing

In some aspects, the presence of the platinum within the CB complexes described herein can introduce specific cytotoxicity. The well-known cytotoxic properties of Pt complexes motivated the exploration of the recognition properties of the CB[8]-secured Pt dimers towards peptides. Prior to the peptide studies herein, the supramolecular system, was not previously demonstrated to bind to proteins. Recent related studies did reveal that thiols, cysteine, and L-glutathione can bind to these supramolecular systems. The studies herein further reveal that the supramolecular CB complex can bind cysteine in a pentamer and trimer peptide. After discovering that a key enzyme in SARS-CoV-2, Mpro (or 3CLpro), is a cysteine protease and can be inhibited by having drugs binding to cysteine and to a neighboring histidine residue, the possible use of the supramolecular systems herein to target these types of enzymes by complexing with the active cysteine145 (Cys145) residue was assessed. The availability and the activity of the cysteine demonstrate this to be a good target for the supramolecular complex. Further, binding the cysteine145 in its active site can greatly inhibit the protease from performing its necessary functions in cleaving the large polyprotein 1a of SARS-CoV-2. The supramolecular complex can further interact with histidine41 (His41) and effectively mask the active site to prevent the protease from performing its necessary viral functions. Further, the presence of platinum can have anti-viral therapeutic benefits.

The Mpro protein is key to control of viral replication and transcription for the severe acute respiratory syndrome coronavirus 2 (SARS-CoV-2) virus, and is therefore an attractive therapeutic target for the development of drugs against COVID-19 infections. Peptidelike anti-HIV-1 drugs, which have been effective at inhibiting SARS-CoV Mpro and HCoV 229E Mpro, are currently being investigated as drug candidates towards SARS-CoV-2 Mpro.

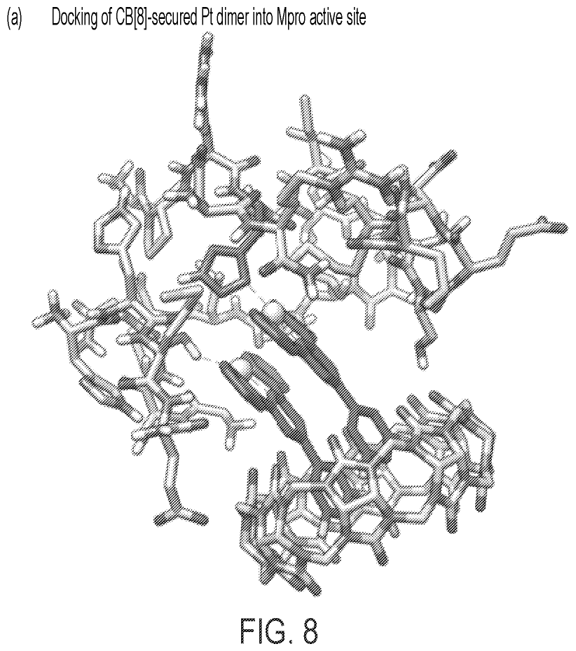

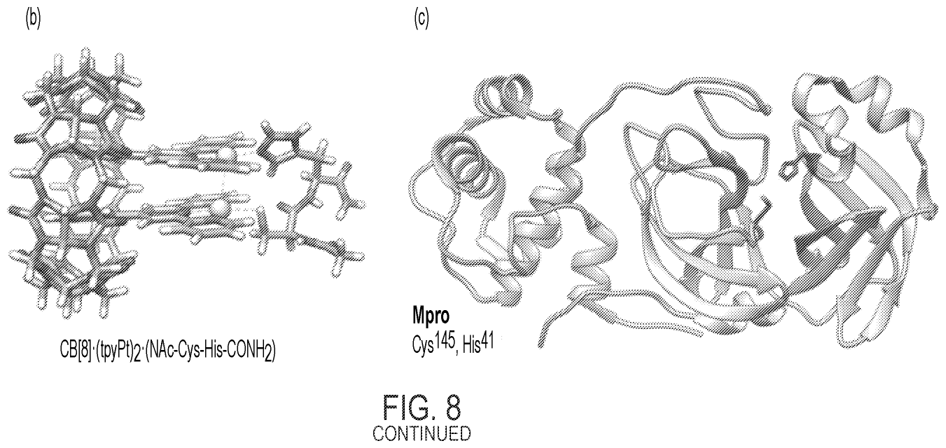

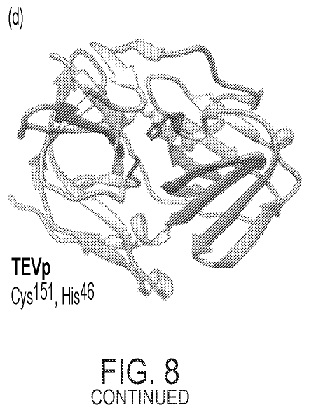

Direct disruption of the catalytic ability of Mpro is possible by sterically blocking the active site, or by inhibiting the activity of the cysteine thiol group or the histidine. The protease acts by cleaving within the Leu-Gln↓(Ser, Ala, Gly) sequence (↓ indicating the cleavage site) of the large polypeptide 1a. Unlike other drug candidates, the CB[8]-secured Pt-tpy complexes can disrupt both units of the catalytic dyad at once by attaching directly to Cys145 and His41 residues; or by binding the Cys145 residue via one Pt center, while the other Pt unit is functionalized with cysteine-containing peptides that enhance affinity and promote selectivity to Mpro. In both cases, the shape of the bulky CB[8]-secured Pt dimer efficiently “plugs” the active site of the enzyme (see FIG. 8, part (a), for a docking sites). Furthermore, several Pt complexes are known antiviral agents. The proximity of the Pt center can further act as an antiviral to SARS-CoV-2.

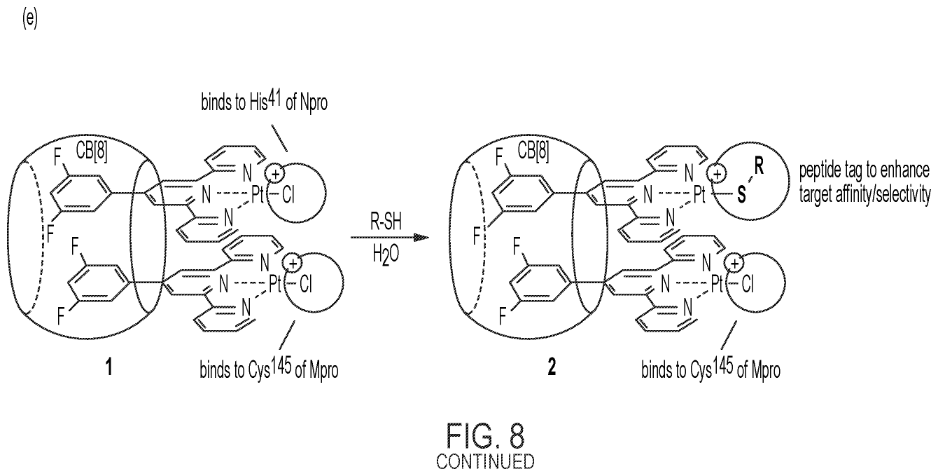

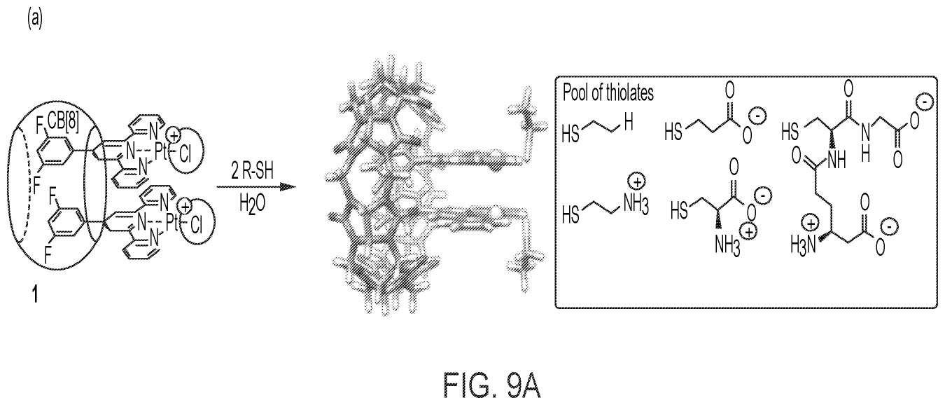

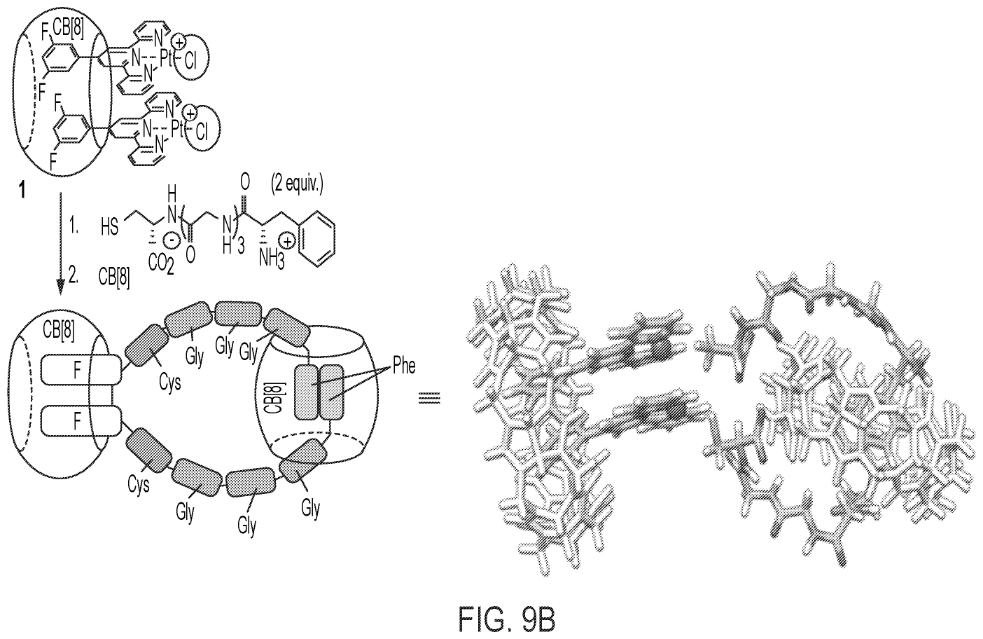

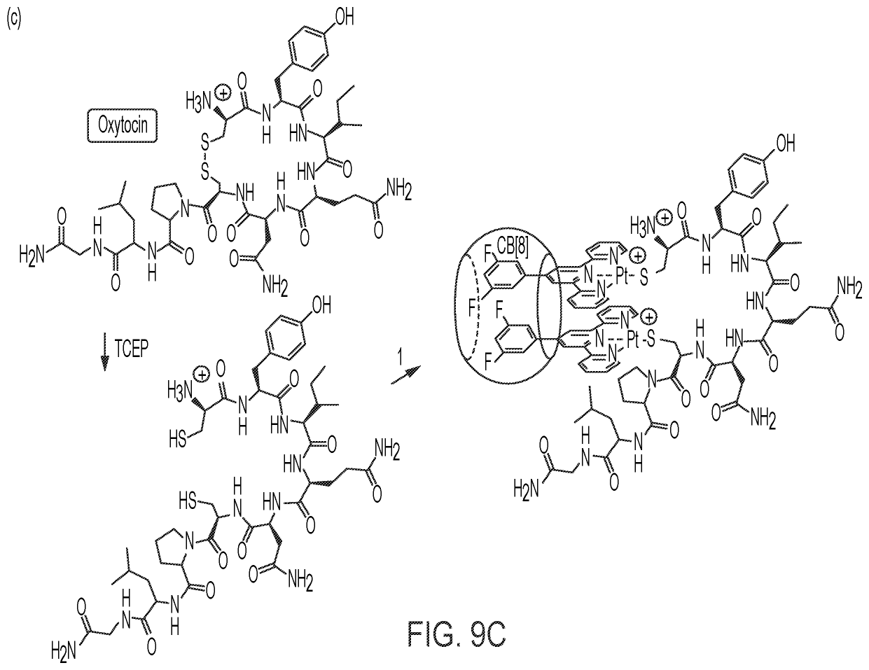

As set forth herein, it has been identified that Pt(II) complexes flanked by 4′ -substituted terpyridine ligands assemble, in aqueous medium, in a stacked, head-to-head motif in the presence of CB[8], with both Platinum centers sitting on top of each other and pointing towards the same direction (see dimer 1 in FIG. 8). As demonstrated herein, it is possible to attach, in situ, quantitatively, and again in aqueous medium, any thiolate ligand to one or both Pt centers, including any cysteine-containing peptide, thereby generating large sets of CB[8]-secured dimers from just a few building blocks (see FIG. 9A). This motif lends itself as particularly useful to designing vast libraries of cytotoxic or target-specific bioactive agents. The section above describes the attachment of peptides to the CB[8]-Pt-tpy secured dimers, and the recognition properties of the Pt/peptide/CB[8] supramolecular assembly towards other CB macrocycles, showing that these sophisticated supramolecular necklaces can be built in situ with just 3 building blocks (see FIG. 9B).

To extend the scope of this motif towards more complex biomolecules (e.g. proteins), the recognition properties of CB[8]-secured Pt dimer 1 towards the well-known cyclic neurohypophyseal nonapeptide oxytocin was assessed (see FIG. 9C), which bears two Cys residues attached to each other via disulfide linkage. Reduction of the first disulfide bond was achieved with opening of the cyclic nonapeptide in situ and in aqueous medium (with the mild reducing agent TCEP (tris(2-carboxyethyl)phosphine). Then without purification, this was directly grafted onto CB[8]-secured Pt dimer 1 (see FIG. 9C). This methodology can be applied to any protein whose surface cysteines are present in oxidized form, in order to alter its enzymatic activity.

The publication of the structure of the key enzyme Mpro in SARS-CoV-2 offers an opportunity to apply this system towards a biological target of utmost importance. Furthermore, Pt dimer 1 is not only capable of binding to the cysteine unit in the active site, but can also bind to both amino acids of the catalytic dyad (Cys145 and His41) as the Pt-Pt distance (3.8 Å) and orientation of the residues are optimum for simultaneous coordination (see FIG. 8).

### EXAMPLES

### CB[8] CB[7] Complex Peptide-Binding

All reagents were purchased from chemical suppliers and used without further purification. Peptide 1 (98% purity) was purchased from KareBay Biochem, Inc., Monmouth Junction, NJ. Cucurbit[7]uril (CB[7]) and Cucurbit[8]uril (CB[8]) were prepared using known procedures. Solvents were of analytical grade and either used as purchased or dried according to procedures described elsewhere. Characterization by Nuclear Magnetic Resonance spectroscopy (NMR) was carried out using a Bruker Ascend 500 MHz spectrometer and a Bruker Avance III HD Ascend 700 MHz located in the Campus Chemical Instrument Center (CCIC) NMR facility at The Ohio State University (OSU).

¹H and ¹³C NMR chemical shifts are reported in parts per million (ppm) and are referenced to TMS using the residual signal of the solvent as an internal reference.

Coupling constants (J) are reported in hertz (Hz). Standard abbreviations used to indicate multiplicity are: s=singlet, d=doublet, t=triplet, q=quartet, m=multiplet.

Ultrahigh resolution/accurate mass measurements were carried out on a Bruker SolariXR 15T Fourier transform ion cyclotron resonance (FT-ICR) instrument. Positively charged ions were generated by electrospray ionization (ESI) and matrix assisted laser desorption/ionization (MALDI) (in the latter, the alpha-cyano hydroxycinnamic acid (HCCA) was used as a matrix).

ESI produced multiply charged ions that were used to confirm product identity. ESI solutions (approx. 5 uM) in acetonitrile/water 1:1 were sprayed by direct infusion.

The resolution of the FT-ICR instrument was set to 300,000 (at m/z 400). For MALDI, a Yag-Nd laser was used (351 nm; 15% of total power).

UV-Vis absorption spectra were recorded on an Agilent HP-8453 diode-array spectrophotometer.

Wavelengths (λ) are reported in nanometers (nm) and molar absorption coefficients (ε) are reported in M⁻¹cm⁻¹.

Semiempirical calculations were carried out with the tight-binding method provided by the GFN2-xTB program on the Owens cluster of the Ohio Supercomputer Center in Columbus, OH (23,392-core Dell Intel Xeon ES-2680 v4 machines).

4′-(3,5-Difluorophenyl)-2,2′:6′,2″-terpyridine and chloro[4′-(3 ,5-difluorophenyl)-2,2′:6′,2″-terpyridine]platinum(II) chloride (PtCl) were prepared according to published procedures.

### Preparation and Characterization of CB[n]-Bound Pt(tpy) Chloride Complexes

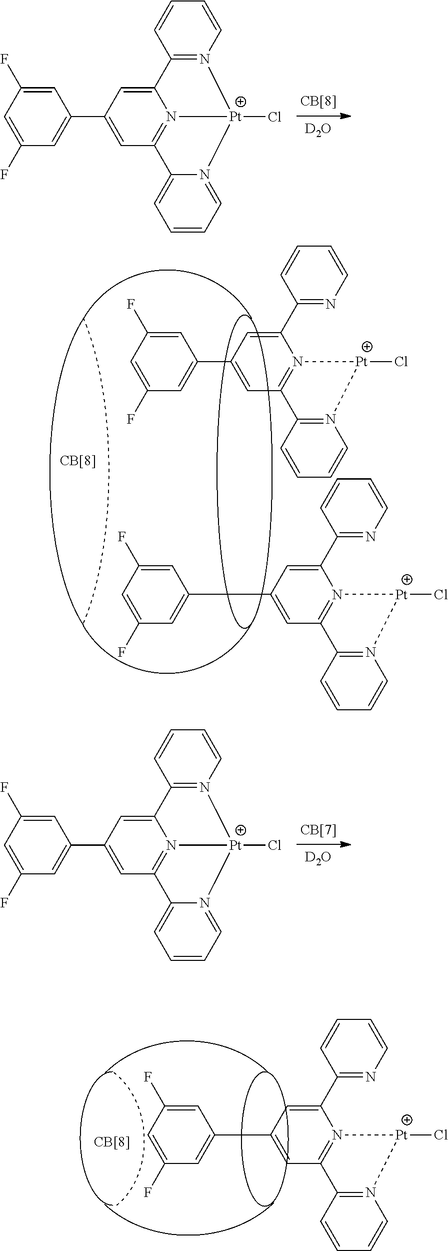

Chloro[4′-(3,5-difluorophenyl)-2,2′:6′,2″-terpyridine]platinum(II) chloride (3.1 mg, 5.1 μmol) was mixed with D₂O (5.0 mL). CB[8] (3.3 mg, 2.5 μmol) or CB[7] (5.9 mg, 5.1 μmol) was added subsequently, and the resulting mixture was sonicated thoroughly. The stock solution was stored at 4° C. for further use.

CB[8]^{Pt}·(Pt·Cl)₂. ¹H NMR (500 MHz, D₂O) δ 8.81 (dd, J=5.7, 1.5 Hz, 4H, H⁶), 8.60 (d, J=8.0 Hz, 4H, H³), 8.19 (td, J=7.8, 1.5 Hz, 4H, H⁵), 8.16 (s, 4H, H^{3′}), 7.59 (ddd, J=7.4, 5.6, 1.3 Hz, 4H, H⁴), 6.64-6.52 (m, 6H, H⁷+H⁸), 5.78 (d, J=15.3 Hz, 8H, H^{CB[8]}), 5.66 (d, J=15.3 Hz, 8H, H^{CB[8]}), 5.40 (s, 16H, H^{CB[8]}), 4.11 (d, J=10.5 Hz, 8H, H^{CB[8]}), 4.08 (d, J=10.5 Hz, 8H, H^{CB[8]}). ¹⁹F NMR (471 MHz, D₂O) δ −106.84.

CB[7]^{Pt}·(Pt·Cl). ¹H NMR (500 MHz, D₂O) δ 9.11 (d, J=8.2 Hz, 4H, H⁶), 8.90 (d, J=5.8 Hz, 4H, H³), 8.59 (s, 4H, H⁵), 8.36 (t, J=7.9 Hz, 4H, H^{3′}), 7.76 (t, J=6.7 Hz, 4H, H⁴), 6.76 (d, J=7.3 Hz, 4H, H⁷), 6.53 (t, J=8.8 Hz, 2H, H⁸), 5.73 (d, J=15.4 Hz, 8H, H^{CB[7]}), 5.63 (d, J=15.3 Hz, 8H, H^{CB[7]}), 5.39 (s, 16H, H^{CB[7]}), 4.10 (t, J=15.8 Hz, 16H, H^{CB[7]}).

### Preparation and Characterization of CB[n]-Bound Pt/Peptide Assemblies

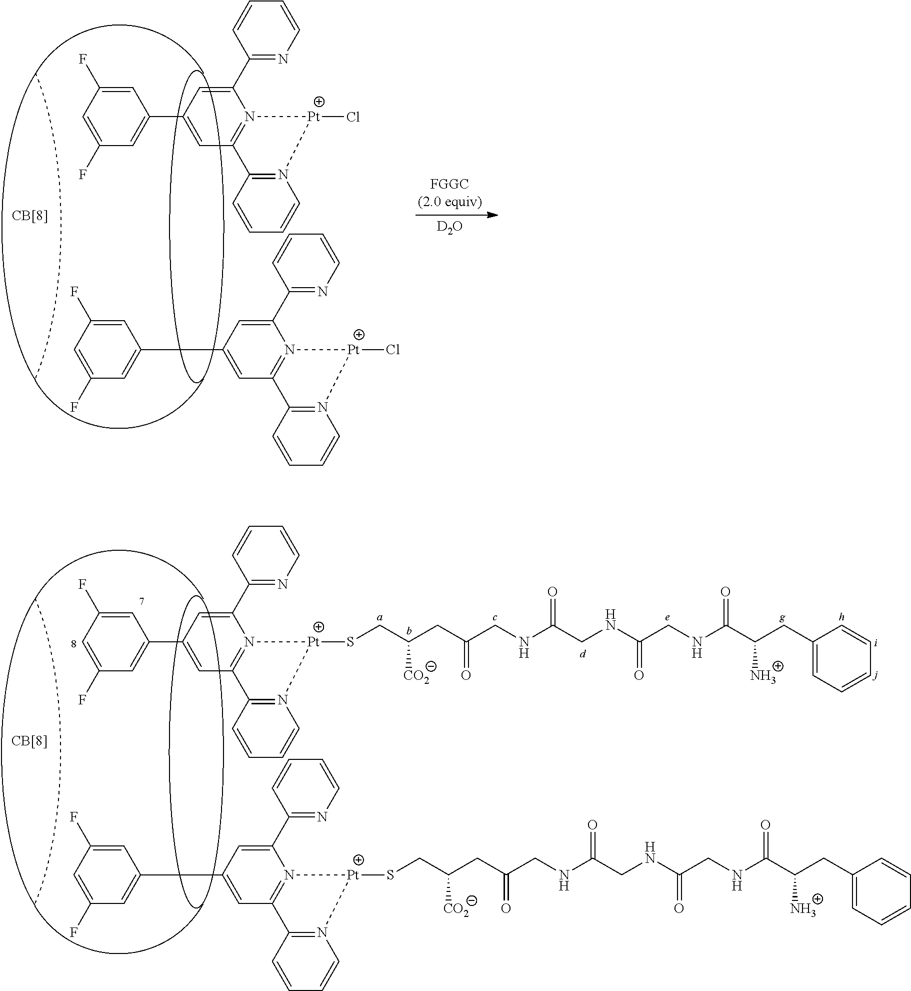

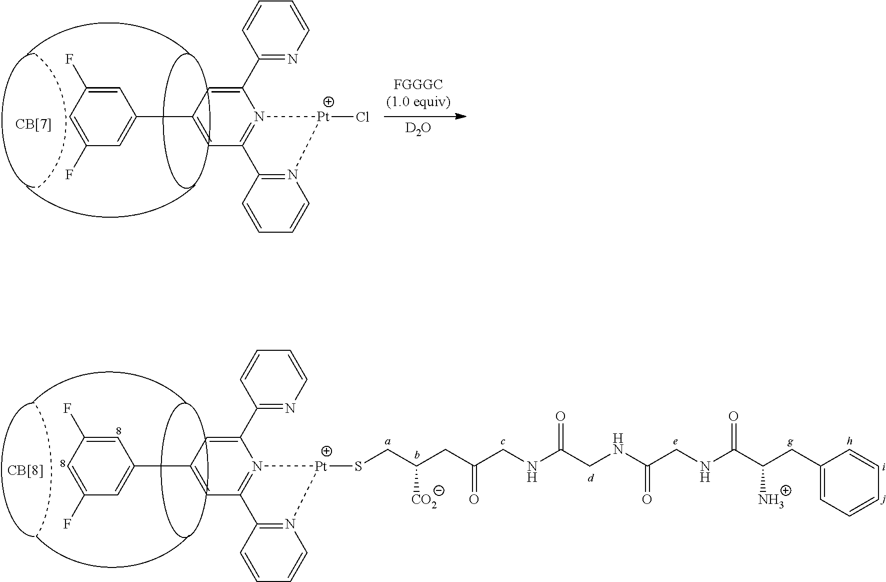

In an NMR tube, stock solutions of complexes CB[8]^{Pt}·(Pt·Cl)₂ or CB[7]^{Pt}·(Pt·Cl) (1.0 mM, 0.60 μmol, 0.60 mL) were treated with an aqueous solution of peptide 1 (50 mM, 0.60 umol, 12 uL) and the mixture was subsequently stirred and kept at 40° C. for 1 h. The color of the sample turned from yellow to red.

Assemblies CB[8]^{Pt}·(Pt·1)₂·CB[7]₂^{Phe}, CB[8]^{Pt}·(Pt·1)₂·CB[8]^{Phe} and CB[7]^{Pt}·(Pt·1)·CB[7]^{Phe} were obtained by adding CB[7] or CB[8] in relevant amounts, and the mixtures were sonicated for 10 minutes.

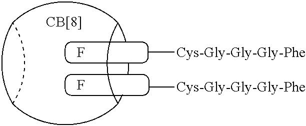

CB[8]^{Pt}·(Pt·1)_{2.} ¹H NMR (500 MHz, D₂O) δ 9.15 (dd, J=10.6, 5.8 Hz, 4H, H⁶), 8.58 (t, J=8.0 Hz, 4H, H³), 8.24-8.10 (m, 8H, H⁵+H^{3′}), 7.58 (dt, J=27.7, 6.6 Hz, 4H, H⁴), 7.42-7.27 (m, 6H, Hⁱ+H^{j}), 7.24 (d, J=7.1 Hz, 4H, H^{h}), 6.67-6.47 (m, 6H, H⁷+H⁸), 5.77 (d, J=15.3 Hz, 8H, H^{CB[8]}), 5.64 (d, J=15.3 Hz, 8H, H^{CB[8]}), 5.38 (s, 16H, H^{CB[8]}), 4.40 (dd, J=7.8, 4.3 Hz, 2H, H^{b}), 4.31-4.20 (m, 2H, H^{f}), 4.07 (t, J=16.5 Hz, 16H, H^{CB[8]}), 3.97-3.83 (m, 8H, H^{c}+H^{e}), 3.76 (q, J=16.8 Hz, 4H, H^{d}), 3.17 (ddt, J=28.7, 13.2, 7.0 Hz, 4H, H^{g}), 3.01-2.67 (m, 4H, H^{a}). ¹³C NMR (126 MHz, D₂O) δ 32.24, 36.77, 42.09, 42.38, 42.49, 53.45, 54.43, 56.58, 71.93, 106.03, 106.15, 109.02, 109.18, 109.24, 121.52, 121.60, 126.24, 126.28, 128.01, 129.02, 129.16, 129.38, 133.72, 137.24, 137.32, 137.40, 142.35, 151.28, 152.44, 152.56, 152.84, 155.79, 155.85, 156.30, 156.66, 156.84, 157.90, 161.90, 162.01, 163.90, 164.00, 169.83, 170.78, 171.38, 171.67, 174.34, 176.65. ¹⁹F NMR (471 MHz, D₂O) δ −107.03. HRMS (ESI): m/z=1644.42682 [M]²⁺ (calcd. 1644.4224 for C₁₂₆H₁₂₄F₄N₄₈O₂₈Pt₂S₂). UV-Vis (H₂O) λ 248 (ε=18.4×10³), 279 (ε=21.4×10³), 317 (ε=12.2×10³), 374 (ε=3.55×10³), 574 (ε=7.10×10²).

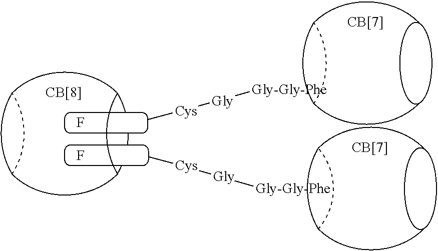

CB[8]^{Pt}·(Pt·1)₂·CB[7]₂^{Phe}. ¹H NMR (500 MHz, D₂O) δ 9.08 (dd, J=20.8, 5.6 Hz, 4H, H⁶), 8.54 (t, J=7.8 Hz, 4H, H³), 8.29-8.00 (m, 8H, H⁵+H^{3′}), 7.67-7.47 (m, 4H, H⁴), 6.60 (s, 2H, H^{j}), 6.56-6.43 (m, 10H, H⁷+H⁸+H^{h}), 6.31 (s, 4H, Hⁱ), 5.70 (d, J=15.3 Hz, 8H, H^{CB[8]}), 5.65-5.52 (m, 36H, H^{CB[8]}+H^{CB[7]}), 5.35 (s, 28H, H^{CB[7]}), 5.34 (s, 16H, H^{CB[8]}), 4.39 (t, J=5.9 Hz, 2H, H^{b}), 4.26 (d, J=10.8 Hz, 2H, H^{f}), 4.19-3.96 (m, 48H, H^{CB[8]}+H^{CB[7]}+H^{c}), 3.94-3.76 (m, 4H, H^{e}), 3.75-3.55 (m, 4H, H^{d}), 2.98 (d, J=15.1 Hz, 2H, H^{a}), 2.90 (d, J=15.1 Hz, 2H, H^{a}), 2.79-2.70 (m, 2H, H^{g}), 2.43 (t, J=13.6 Hz, 2H, H^{g}). ¹³C NMR (126 MHz, D₂O) δ 174.22, 172.47, 171.64, 170.57, 170.35, 163.94, 162.03, 157.91, 156.88, 156.70, 156.25, 152.82, 152.44, 151.31, 142.42, 137.32, 133.66, 129.10, 128.00, 127.30, 126.35, 121.65, 109.23, 109.04, 106.20, 105.97, 71.95, 71.14, 56.46, 54.90, 53.48, 52.50, 43.48, 42.49, 41.93, 36.08, 32.09. ¹⁹F NMR (471 MHz, D₂O) δ −106.91. HRMS (ESI): m/z=1403.38324 [M+2H]⁴⁺ (calcd. 1403.38286 for C₂₁₀H₂₀₈F₄N₁₀₄O₅₆Pt₂S₂). UV-Vis (H₂O) λ 248 (ε=20.1×10³), 279 (ε=23.6×10³), 318 (ε=12.1×10³), 375 (ε=3.78×10³), 541 (ε=8.30×10²).

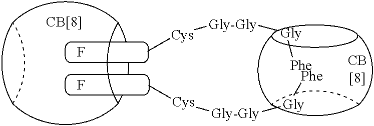

CB[8]^{Pt}·(Pt·1)₂·CB[8]^{Phe}. ¹H NMR (500 MHz, D₂O) δ 9.29 (d, J=5.6 Hz, 2H, H⁶), 9.19 (d, J=5.5 Hz, 2H, H⁶), 8.70 (d, J=8.0 Hz, 2H, H³), 8.66 (d, J=8.0 Hz, 2H, H³), 8.41-8.10 (m, 8H, H⁵+H^{3′}), 7.70 (q, J=8.2, 7.7 Hz, 4H, H⁴), 6.83-6.61 (m, 8H, H⁷+H⁸+H^{j}), 6.56-6.36 (m, 4H, Hⁱ), 6.03 (d, J=7.6 Hz, 4H, H^{h}), 5.86 (d, J=15.3 Hz, 24H, H^{CB[8]}), 5.74 (d, J=15.3 Hz, 8H, H^{CB[8]}), 5.64 (s, 16H, H^{CB[8]}), 5.48 (s, 16H, H^{CB[8]}), 4.53 (t, J=6.2 Hz, H^{b}), 4.43 (d, J=17.3 Hz, H^{f}), 4.30 (d, J=15.2 Hz, 16H, H^{CB[8]}), 4.23-4.11 (m, 24H H^{CB[8]}+H^{c}+H^{e}), 4.01-3.85 (m, 4H, H^{d}), 3.10-3.00 (m, 2H, H^{a}), 2.97-2.87 (m, 2H, H^{a}), 2.39 (d, J=13.0 Hz, 2H, H^{g}), 2.25 (t, J=13.2 Hz, 2H, H^{g}). ¹³C NMR (176 MHz, D₂O) δ 174.22, 172.30, 171.77, 170.59, 170.24, 163.74, 163.67, 162.31, 162.24, 157.90, 157.87, 156.91, 156.89, 156.81, 156.78, 156.76, 156.69, 153.90, 152.95, 152.90, 152.86, 152.46, 151.26, 142.76, 142.40, 137.32, 132.78, 129.14, 129.06, 128.95, 127.77, 126.64, 126.33, 126.25, 121.60, 121.49, 109.21, 109.07, 106.33, 106.18, 106.03, 72.04, 71.95, 56.38, 54.97, 53.62, 53.47, 43.26, 42.63, 41.97, 37.16, 32.01. ¹⁹F NMR (471 MHz, D₂O) δ −107.06. HRMS (ESI): m/z=1154.30735 [M+2H]⁴⁺ (calcd. 1154.30919 for C₁₇₄H₁₇₂F₄N₈₀O₄₄Pt₂S₂); m/z=1538.73924 [M+H]³⁺ (calcd. 1538.74316 for C₁₇₄H₁₇₁F₄N₈₀O₄₄Pt₂S₂). UV-Vis (H₂) λ 249 (ε=18.4×10³), 279 (ε=22.5×10³), 317 (ε=12.3×10³), 375 (ε=3.66×10³), 544 (ε=9.13×10²).

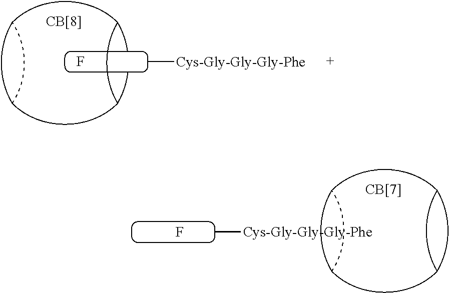

CB[7]^{Pt}·(Pt·1). ¹H NMR (500 MHz, D₂O) δ 8.82 (s, 2H, H⁶), 8.27 (s, 2H, H³), 8.16 (s, 4H, H⁵+H^{3′}), 7.57 (s, 2H, H⁴), 7.25 (s, 2H, H⁷), 7.01 (s, 1H, H⁸), 6.72 (s, 1H, H^{j}), 6.64 (s, 2H, Hⁱ), 6.50 (s, 2H, H^{h}), 5.58 (d, J=15.4 Hz, 14H, H^{CB[7]}), 5.34 (s, 14H, H^{CB[7]}), 4.34 (s, 1H, H^{b}), 4.20 (d, J=10.8 Hz, 1H, H^{f}), 4.04 (d, J=15.4 Hz, 14H, H^{CB[7]}), 3.98-3.88 (m, 2H, H^{c}), 3.77 (q, J=17.5, 17.0 Hz, 2H, H^{e}), 3.66-3.47 (m, 2H, H^{d}), 2.90 (dd, J=47.7, 14.5 Hz, 2H, H^{g}), 2.60 (dt, J=56.2, 13.0 Hz, 2H, H^{a}). ¹³C NMR (176 MHz, D₂O) δ 174.45, 172.10, 171.42, 170.33, 170.31, 164.07, 164.00, 162.66, 162.58, 157.80, 156.23, 153.36, 152.42, 151.19, 142.51, 137.66, 133.64, 129.33, 128.28, 127.82, 127.66, 125.72, 110.57, 71.13, 56.84, 54.72, 52.48, 43.13, 42.48, 41.87, 36.28, 31.81. ¹⁹F NMR (471 MHz, D₂O) δ −107.86. HRMS (ESI): m/z=1071.28488 [M+H]²⁺ (calcd. 1071.28463 for C₈₁H₈₀F₂N₃₆O₂₀Pt₁S₁). HRMS (MALDI): m/z=2141.569 [M]⁺ (calcd. 2141.562 for C₈₁H₇₉F₂N₃₆O₂₀Pt₁S₁). UV-Vis (H₂O) λ 250 (ε=23.5×10³), 289 (ε=27.4×10³), 348 (ε=8.54×10³), 500 (ε=1.06×10³).

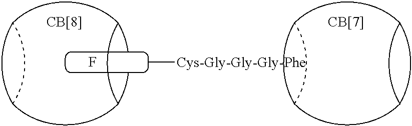

CB[7]^{Pt}·(Pt·1)·CB[7]^{Phe}. ¹H NMR (500 MHz, D₂O) δ 9.19 (d, J=4.6 Hz, 2H, H⁶), 9.15 (d, J=8.0 Hz, 2H, H³), 8.67 (s, 2H, H^{3′}), 8.40 (t, J=7.7 Hz, 2H, H⁵), 7.79 (t, J=6.8 Hz, 2H, H⁴), 6.76 (d, J=7.1 Hz, 2H, H⁷), 6.65 (t, J=7.5 Hz, 1H, H⁸), 6.58-6.45 (m, 3H, Hⁱ+H^{j}), 6.34 (d, J=7.5 Hz, 2H, H^{h}), 5.73 (d, J=15.4 Hz, 7H, H^{CB[7]}), 5.67 (d, J=15.5 Hz, 7H, H^{CB[7]}), 5.62 (dd, J=15.2, 4.3 Hz, 14H, H^{CB[7]}), 5.40 (d, J=4.8 Hz, 28H, H^{CB[7]}), 4.57-4.53 (m, 1H, H^{b}), 4.39-4.29 (m, 1H, H^{f}), 4.17 (s, 2H, H^{c}), 4.15-4.03 (m, 28H, H^{CB [7]}), 4.02-3.57 (m, 4H, H^{d}+H^{e}), 3.04 (d, J=13.0 Hz, 2H, H^{a}), 2.98-2.89 (m, 1H, H^{g}), 2.58-2.43 (m, 1H, H^{g}). ¹³C NMR (176 MHz, D₂O) δ 174.26, 172.56, 171.63, 170.56, 163.72, 163.64, 162.30, 162.22, 159.85, 156.36, 156.29, 156.21, 156.14, 153.46, 151.77, 142.18, 139.45, 133.65, 128.54, 127.98, 127.94, 127.53, 127.23, 127.19, 126.63, 123.70, 109.31, 109.28, 109.18, 109.15, 103.93, 71.18, 71.13, 71.11, 55.85, 54.89, 52.53, 52.50, 52.47, 46.51, 43.55, 42.47, 42.00, 36.06. ¹⁹F NMR (471 MHz, D₂O) δ −109.96. HRMS (ESI): m/z=1652.45424 [M+H]²⁺ (calcd. 1652.45652 for C₁₂₃H₁₂₂F₂N₆₄O₃₄Pt₁S₁). UV-Vis (H₂O) λ 260 (ε=24.0×10³), 289 (ε=31.4×10³), 345 (ε=10.6×10³), 484 (ε=1.02×10³).

### Cysteine-Protease Binding

The Mpro protein, as well as papain 19 and TEVp cysteine proteases can be assessed to see how the Pt-CB[8] complex can both bind the active cysteine residues, but also affect activity. The presence of the Pt in proximity to the active protein can further provide details concerning the anti-viral properties of the complex. The binding mode and quantify the affinity of CB[8]-secured Pt chloride dimer 1 towards short model peptides that contain both Cys and His residues and mimic the geometry of the active site of Mpro can be first assessed (see FIG. 8b for an optimized structure of assembly 1 attached to the dipeptide NAc-Cys-His-CONH2). We can then quantify the inhibitory activity of dimer 1 towards papain19 and Tobacco Etch Virus protease (TEVp) which are readily available cysteine proteases with active sites that mimic Mpro (FIGS. 8c and 8d).

It is therefore possible to rationally design, through the use of docking techniques, a series of cysteine-containing peptide tags that can be grafted onto one of the Platinum units to form CB[8]-secured Pt heterodimers 2 (see FIG. 8e). The Pt chloride unit can bind to Cys145 in the active site, and the Pt/peptide unit will enhance affinity and promote selective binding to Mpro. Papain and TEVp can be used again as Mpro mimics to test this methodology experimentally. In parallel, docking experiments can be carried out to identify peptide tags selective for SARS-CoV-2 Mpro. Using papain as a model enzyme can also advantageously identify peptide tags to target PLpro, a papain-like protease also involved in SARS-CoV-2 replication.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A cucurbituril peptide complex, comprising:
at least one platinum-terpyridine-peptide complex according to formula (I): 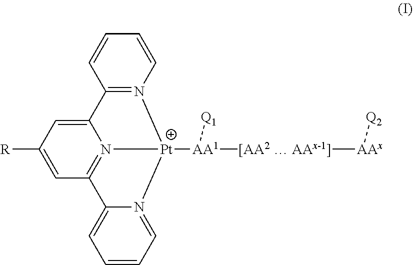
where:
R is phenyl or substituted phenyl;
each AA is an amino acid of a peptide;
AA¹ is an amino acid bound to a platinum metal center;
AA^{X} is a terminal amino acid of the peptide;
x is from 2 to 5000; and
Q₁ and Q₂ are independently either non-existent or at least one additional amino acid of the peptide; and
at least one curcurbituril CB[n], where n is from 5 to 10,
wherein:
each curcurbituril CB[n] is circumposed about a head portion or a tail portion of the at least one platinum-terpyridine-peptide complex;
the head portion of the at least one platinum-terpyridine-peptide complex comprises group R of the at least one platinum-terpyridine-peptide complex; and
the tail portion of the at least one platinum-terpyridine-peptide complex comprises the terminal amino acid AA^{X}.

2. The curcurbituril peptide complex of claim 1, comprising two platinum-terpyridine-peptide complexes and one curcurbituril CB[n], in which the curcurbituril CB[n] is circumposed about both head portions of the two platinum-terpyridine-peptide complexes.

3. The curcurbituril peptide complex of claim 1, comprising two platinum-terpyridine-peptide complexes and three CB[n], in which a first CB[n] is circumposed about both head portions of the two platinum-terpyridine-peptide complexes, a second CB[n] is circumposed about the tail portion of one of a first of the two platinum-terpyridine-peptide complexes, and a third CB[n] is circumposed about the tail portion of a second of the two platinum-terpyridine-peptide complexes.

4. The curcurbituril peptide complex of any of the preceding claims, wherein each AA¹ is a cysteine or histidine residue.

5. The curcurbituril peptide complex of any of the preceding claims, wherein Q is non-existent.

6. The curcurbituril peptide complex of any of the preceding claims, wherein each R is a methylphenyl (tolyl), a phenol, or a halophenyl.

7. The curcurbituril peptide complex of any of the preceding claims, wherein each R is a chlorinated phenyl, a dichlorinated phenyl, a fluorinated phenyl, or a difluorinated phenyl.

8. The curcurbituril peptide complex of any of the preceding claims, wherein each R is 3,5-difluorophenyl.

9. The curcurbituril peptide complex of any of the preceding claims, wherein each curcurbituril is CB[7] or CB[8].

10. The curcurbituril peptide complex of any of claims 2 to 9, wherein the each peptide is identical in amino acid sequence.

11. The curcurbituril peptide complex of any of the preceding claims, wherein each R is identical.

12. The curcurbituril peptide complex of any of the preceding claims, wherein x is from 2 to 5.

13. The curcurbituril peptide complex of any of the preceding claims, wherein each terminal amino acid AA^{X} is phenylalanine or tyrosine.

14. A method for preparing the curcurbituril peptide complex of any of the preceding claims, the method, comprising conjugating the peptide to the at least one platinum-terpyridine-peptide complex and subsequently adding an equivalent of the CB[n] thereto.

15. The method of claim 14, further comprising adding a further equivalent of CB[8] or CB[7].

16. The method of claim 15, wherein the further equivalent is about 0.5 times the concentration of the at least one platinum-terpyridine-peptide complex.

17. The method of claim 15, wherein the further equivalent is of about 2 time the concentration of the at least one platinum-terpyridine-peptide complex.

18. The method of claim 14, wherein the first peptide conjugates to the at least one platinum-terpyridine-peptide complex by displacing a halogen ligand.

19. A method of binding a peptide or a protein, the method comprising incubating a curcurbituril peptide complex according to any of claims 1 to 13 with a target peptide in a solution, wherein the peptide comprises at least one cysteine residue.

20. The method of claim 19, wherein the target peptide comprises Mpro of SARS-CoV-2.

21. The method of claim 19 or 20, wherein the solution is in vitro.

22. The method of claim 19 or 20, wherein the solution is in vivo.

23. A complex comprising a cucurbituril peptide complex according to any of claims 1 to 13 and a cysteine protease.

24. The complex of claim 23, wherein the cysteine protease is MPro of severe acute respiratory syndrome coronavirus 2 (SARS-CoV-2).
